(19) United States
(12) Patent Application Publication
(10) Pub. No.: US 2023/0321008 A1
(43) Pub. Date: Oct. 12, 2023

(54) INHIBITORS OF ACID SPHINGOMYELINASE FOR PREVENTING AND TREATING THE COVID-19 DISEASE

(71) Applicants: ASSISTANCE PUBLIQUE - HOPITAUX DE PARIS, PARIS (FR); UNIVERSITE DE PARIS, PARIS (FR)

(72) Inventors: Nicolas HOERTEL, ISSY LES MOULINEAUX (FR); Frederic LIMOSIN, PARIS (FR); Marina Lucia SANCHEZ RICO, ISSY LES MOULINEAUX (FR); Miriam ABELLAN, Madrid (ES); Pedro DE LA MUELA, MADRID (ES); Erich GULBINS, Essen (DE); Johannes KORNHUBER, NURNBERG (DE); Alexander CARPINTEIRO, Essen (DE)

(21) Appl. No.: 18/002,814

(22) PCT Filed: Jun. 24, 2021
(86) PCT No.: PCT/EP2021/067427

§ 371 Date: Dec. 21, 2022

Related U.S. Application Data
Application No. PCT/EP2021/067427 is a continuation-in-part of application No. 17/146,013, filed on Jan. 11, 2021.

(30) Foreign Application Priority Data
Jun. 24, 2020 (EP) 20305698.1
Sep. 29, 2020 (EP) 20306117.1
Feb. 12, 2021 (EP) 21305188.1

(51) Int. Cl. A61K 31/136; A61K 31/4965; A61P 31/14; A61K 31/135; A61K 31/15

(52) U.S. Cl. CPC A61K 31/136; A61K 31/135; A61K 31/15; A61K 31/4965; A61P 31/14

(57) ABSTRACT

The present invention relates to the preventive and therapeutic uses of acid sphingomyelinase inhibitors (FIASMAs) such as psychotropic medications and non-psychotropic compounds having FIASMA activity, for lowering the risk of death and/or intubation in patient suffering from a viral infection caused by at least one betacoronavirus, in particular by the SARS-CoV-2 (Severe Acute Respiratory Syndrome Coronavirus 2).

### SUMMARY OF THE INVENTION

The present invention relates to the preventive and therapeutic uses of acid sphingomyelinase inhibitors (FIASMAs) such as psychotropic medications and non-psychotropic compounds having FIASMA activity, for lowering the risk of death and/or intubation in patient suffering from a viral infection caused by at least one betacoronavirus, in particular by the SARS-CoV-2 (Severe Acute Respiratory Syndrome Coronavirus 2) through reduced cell and blood concentration of ceramides.

### BACKGROUND OF THE INVENTION

Global spread of the novel coronavirus SARS-CoV-2, the causative agent of coronavirus disease 2019 (COVID-19), has created an unprecedented infectious disease crisis worldwide [1-3].

SARS-CoV-2 belongs to the species Coronavirus, in the genus Betacoronavirus and family Coronaviridae. Coronaviruses are enveloped viruses with a helically symmetrical capsid. They have a single-stranded, positive-sense RNA genome and are capable of infecting cells in birds and mammals. The morphology of the virions is typical, with a halo of protein protuberances (‘Spike’) which gave them their name of ‘crown virus’. Among the four genera of coronaviruses, the Betacoronavirus genus (β-CoVs or Beta-CoVs), comprising virus infecting animals and/or humans, is subdivided into four lineages designated as A, B, C and D:
Lineage A also designated as subgenus Embecovirus includes HCoV-OC43 and HCoV-HKU1, virus able to infect various species;
Lineage B also designated as subgenus Sarbecovirus includes SARS-CoV-1, SARS-CoV-2, and Bat SL-CoV-WIV1;
Lineage C also designated as subgenus Merbecovirus includes Tylonycteris bat coronavirus HKU4 (BtCoV-HKU4), Pipistrellus bat coronavirus HKU5 (BtCoV-HKU5), and MERS-CoV, able to infect notably camels and humans;
Lineage D also designated as subgenus Nobecovirus includes Rousettus bat coronavirus HKU9 (BtCoV-HKU9).

The Betacoronaviruses of the greatest clinical importance concerning humans are:
OC43 and HKU1 of the A lineage,
SARS-CoV-1 and SARS-CoV-2 of the B lineage, and
MERS-CoV of the C lineage.

In humans, coronavirus infections can cause respiratory pathologies associated with symptoms similar to the common cold, bronchiolitis and more serious diseases such as the Severe Acute Respiratory Syndrome caused by SARS-CoV-1, which generated an epidemic in 2003, and the Middle Eastern Respiratory Syndrome caused by MERS-CoV, which generated an epidemic in 2012. SARS-CoV-2 is the betacoronavirus causing the coronavirus epidemic of 2019-2021, generating the form of pneumonia known as coronavirus disease 2019 or COVID-19.

Symptoms of infection with SARS-CoV-2 are roughly similar to those of seasonal influenza infections: they include fever, fatigue, dry cough, shortness of breath, difficult breathing, pneumonia, renal failure, and may lead to death in severe cases.

The severity of clinical signs requires that approximately 20% of patients remain in hospital and 5% require admission to intensive care. However, this case-fatality rate remains uncertain, due to the difficulty in estimating the number of confirmed cases and deaths directly attributable to SARS-CoV-2 infection in the field. The most serious forms are observed in people who are vulnerable because of their age (over 70) or associated diseases such as hypertension, diabetes and/or coronary heart disease.

The majority of treatments currently received by COVID-19 patients are primarily aimed at alleviating the symptoms of fever, cough and dyspnea in order to promote spontaneous recovery. Yet, no efficient treatment has ever been proposed to prevent intubation or death of the patient once they are in a severe stage of the disease.

Although the availability of vaccines has raised hope for a decline of the pandemic, the search for an effective treatment for patients with COVID-19 among all available medications is still urgently needed.

### DESCRIPTION OF THE INVENTION

In this context, the present inventors have discovered that the use of some antidepressants and non-psychotropic compounds (in particular hydroxyzine), all of which being able to inhibit the acid sphingomyelinase (ASM) / ceramide system (FIASMA agents), were significantly associated with reduced mortality in patients hospitalized for COVID-19 (see examples 1-3 below).

As short-term use of most FIASMA medications is generally well-tolerated at their usual doses for their respective clinical indications [10, 11], it has been hypothesized by the present inventors that modulating the ASM/ceramide system may provide a potent medication for preventing or treating COVID-19.

To test this hypothesis, the present inventors were the first to set up a large observational multicenter retrospective study on human beings, to show the potential usefulness of FIASMA psychotropic medication such as fluoxetine or fluvoxamine at usual antidepressant dose among patients with and without mental disorder hospitalized for COVID-19. Their in vivo observational results are statistically very strong, and they will have a huge impact on the management of the world-wide current health crisis.

The mechanisms underlying these observations have been afterwards explained on a molecular level: as a matter of fact, it has been recently demonstrated that SARS-CoV-2 activates the acid sphingomyelinase (ASM)/ceramide system, resulting in the formation of ceramide-enriched membrane domains that serve viral entry and infection by clustering ACE2, the cellular receptor of SARS-CoV-2 and influence viral intracellular traffic [4]. This in vitro study [4] showed that several FIASMA (Functional Inhibitors of Acid SphingomyelinAse) antidepressant medications, including amitriptyline, imipramine, desipramine, fluoxetine, sertraline, escitalopram and maprotiline ([5]), inhibited ASM and the formation of ceramide-enriched membrane domains, and prevented Vero cells from being infected with SARS-CoV-2. Reconstitution of ceramide in cells treated with these FIASMA antidepressant medications restored infection with SARS-CoV-2. Oral use of the FIASMA antidepressant and FIASMA amitriptyline in healthy volunteers also efficiently blocked infection of freshly isolated nasal epithelial cells with SARS-CoV-2 [4]. These preclinical data were confirmed by another study that demonstrated an inhibition of the infection of cultured epithelial cells with SARS-CoV-2 by the FIASMA antidepressant fluoxetine [6].

Findings from other recent clinical studies are also consistent with our results: a randomized double-blind controlled study [7] showed also significant protective effects on COVID-19 disease progression of the antidepressant and FIASMA fluvoxamine. Second, another observational multicenter retrospective study showed that use of other antidepressants, all of which having a FIASMA activity, were significantly associated with reduced mortality in patients hospitalized for COVID-19 [8, 9].

It is noteworthy that, due to the pandemic situation experienced by the scientific community for more than one year, a huge number of in vitro studies, hypothesis and/or ideas have been published in order to try to reduce the SARS COV-2 infectivity or lethal symptoms. Yet, there was a big gap between some encouraging results in preclinical studies, including in vitro studies, and the non-significant effect on mortality observed in patients hospitalized with COVID-19 for several molecules, such as hydroxychloroquine ([51]), remdesivir ([52]), and azithromycin ([53]). Therefore, the skilled person is nowadays not ready to accept all and every explanations and potential treatments that have been proposed in the art, except when supported with strong data, as it is the case in the present invention. The skilled person has also understood that the mechanisms involved in the infectivity of other coronaviruses (such as MHV) can not be transposed to SARS COV-2, which is a much more complex virus, notably in terms of cell entry, recognition, and number of variants (cf. [54] - [56]).

### Definitions

In the context of the invention, the term “betacoronavirus” designates any virus belonging to the Betacoronavirus genus (β-CoVs or Beta-CoVs), in particular any betacoronavirus belonging to one of the four lineages designated as A, B, C and D. It designates a betacoronavirus infecting animals and/or humans. In particular, this designation includes the betacoronaviruses infecting human organisms chosen among OC43, HKU1, SARS-CoV-1, SARS-CoV-2 and MERS-CoV.

The term “viral infection due to at least one betacoronavirus” designates the fact that host cells of an organism have been infected by at least one betacoronavirus, the whole organism being said to have a viral infection.

A betacoronavirus infection in humans is usually diagnosed by a healthcare professional, based on observation of the infected patient’s symptoms. Additional biological tests may be required to confirm the diagnosis: blood and/or sputum and/or bronchoalveolar fluid tests. Infection by a betacoronavirus can be established, for example, by molecular biological detection and/or viral titration of respiratory specimens, or by assaying blood for antibodies specific for said betacoronavirus. Conventional diagnostic methods comprise techniques of molecular biology such as PCR, which is well known to the person in the field.

As used herein, the term “treatment” refers to fighting the betacoronavirus infection in a human or animal organism. By administering the agent of the invention, the symptoms associated with the betacoronovirus infection (respiratory syndrome, etc.) will be reduced, or the severity of the disease will decrease. It is also possible, with the treatment of the invention, to prevent the worsening of the disease and also to prevent, in certain cases, the viral infection itself (i.e., the entry of the virus into the patient cells).

In the context of the invention, the terms “COVID-19 disease” mean the disease linked to the infection with the SARS-CoV-2 (Severe Acute Respiratory Syndrome Coronavirus 2).

In the context of the invention, a “symptomatic COVID-19 disease” is characterized by a patient who shows at least one symptom of the COVID-19 disease. The most common symptoms of the COVID-19 disease are fever, muscle aches, headaches, fatigue, loss of taste and smell and respiratory symptoms such as a dry cough, difficulty breathing and a lack of oxygen. A symptomatic disease is in contrast to an asymptomatic disease which is characterized by a patient who is a carrier for a disease or infection but experiences no symptoms.

In particular, for the betacoronavirus disease (such as COVID-19), the following forms are usually observed:
an “asymptomatic form” wherein the subject is a carrier of at least on betacoronavirus but shows no symptom.
a “mild COVID form” wherein the subject shows the first symptoms (or signs) of a COVID disease, as listed above. Mild COVID symptoms (such as COVID-19) comprise e.g., mild dry cough, mild muscle pain, mild headache, mild fever, mild fatigue, loss of taste or smell.
a “strong COVID form” wherein the subject shows strong respiratory symptoms such as difficulty breathing, lack of oxygen; other stronger symptoms such as fever, dry cough, aches and pains, nasal congestion, strong headache, conjunctivitis, sore throat, skin rash, discoloration of fingers or feet, or any combination thereof; as well as a deterioration of the general state of health with frequent diarrhoea, but also liver or urinary disorders, dizziness or neuromuscular problems; some of these symptoms may require hospitalisation. Most patients have an abnormal chest X-ray or CT scan within the first few days of illness, even in the absence of respiratory signs.
A “severe COVID form” or “critical COVID form” or “aggressive COVID form” wherein the subject has life-threatening symptoms (or signs) of COVID (e.g. COVID-19), comprising at least one selected from (but not limited to): respiratory distress, lung disorders, liver disorders, kidney disorders, neuromuscular disorders, brain disorders, etc, that requires hospitalisation and/or intensive care (in intensive care units (ICU)).

In the context of the invention, the “severe symptomatic COVID-19 disease” is characterized by severe symptoms of the COVID-19 disease, in particular respiratory symptoms. Severe symptoms are acute respiratory distress syndrome that requires hospitalization of the patient in any unit, and especially in the intensive care unit (ICU) in case of critical infection. In the context of the invention, the abbreviation “ICU” refers to an intensive care unit, a special department of a hospital or health care facility that provides intensive treatment medicine.

In the context of the invention, “COVID-19 patients” are human patients infected with the SARS-CoV-2 (Severe Acute Respiratory Syndrome Coronavirus 2).

By “antidepressant agent”, it is herein meant an active agent that is capable to treat mood disorders, such as depression (including severe depression) and/or dysthymia, and/or anxiety disorders. Antidepressant agents according to the invention include, without limitation, serotonin reuptake inhibitors (SRIs); tricyclic antidepressants (TCAs); monoamine oxidase inhibitors (MAOs).

Serotonin reuptake inhibitors (SRIs) designate a class of compounds that typically act by inhibiting the reuptake of the serotonin neurotransmitter into the presynaptic terminal, thereby increasing the serotonin extracellular level and thus serotoninergic transmission. Such compounds can act selectively or non-selectively on the neurotransmitter serotonin. SRIs can indeed also display various degrees of selectivity towards the other monoamine reuptake systems, in particular the transporters for norepinephrine and dopamine.

SRIs typically include selective serotonin reuptake inhibitors (SSRIs), serotonine and norepinephrine reuptake inhibitors (SNRIs) and serotonin-norepinephrine-dopamine reuptake inhibitor (SNDRIs).

Examples of selective serotonin reuptake inhibitors (SSRIs) include, without limitation, fluoxetine, citalopram, escitalopram, sertraline, norsertraline, paroxetine, fluvoxamine, vortioxetine, femoxetine, indalpine, alaproclate, cericlamine, ifoxetine, zimelidine, dapoxetine, and etoperidone. In a preferred embodiment, they are chosen among citalopram, escitalopram, sertraline, norsertraline, paroxetine, fluvoxamine, vortioxetine, femoxetine, indalpine, alaproclate, cericlamine, ifoxetine and zimelidine.

Examples of active SSRIs metabolites include, without limitation, desmethylcitalopram, didesmethylcitalopram, and seproxetine (i.e. (S)-norfluoxetine).

Examples of serotonine and norepinephrine reuptake inhibitors (SNRIs) include, without limitation, duloxetine, venlafaxine, desvenlafaxine, milnacipran, levominalcipran and sibutramine.

Examples of serotonin-norepinephrine-dopamine reuptake inhibitor (SNDRIs) (also known as triple reuptake inhibitor or TRI) include, without limitation, bicifadine, brasofensine, tesofensine and nomifensine.

Examples of tricyclic antidepressants (TCAs) include, without limitation, clomipramine, amoxapine, nortriptyline, maprotiline, trimipramine, imipramine, desipramine and protriptyline.

Examples of monoamine oxidase inhibitors (MAOs) include, without limitation, iproniazide, phenelzine, tranylcipromine, moclobemide, selegiline and rasagiline.

Other suitable antidepressant agents are for example: anpirtoline hydrochloride, CGS-12066A, CGS 12066B dimaleate, oxymetazoline, 5-carboxamidotryptamine, CP-93129 and salts thereof (such as CP-93129 dihydrochloride), CP-94253 and salts thereof (such as CP-94253 hydrochloride), CP-122,288, CP-135,807, RU-24969 and salts thereof (such as RU-24969 hemisuccinate), ziprasidone, asenapine, 5-nonyloxytryptamine oxalate, pindolol and (S)-(-)-pindolol and the like.

Antidepressant agent can act by blocking presynaptic alpha-2 adrenergic receptors. Such blockers include, among others, mirtazapine, aptazapine, esmirtazapine, setiptiline and S32212 (also known as N-[4-methoxy-3-(4-methylpiperazin-1-yl)phenyl]-1,2-dihydro-3H-benzo[e]indole-3-carboxamide).

Antidepressant agent can also be atypical antidepressant (defined as such as they do not belong to any of the foregoing classes of antidepressants), for example, without limitation, tianeptine, agomelatine, nefazodone, trazodone, buspirone, tandospirone, and ketamine.

Antidepressant agent can also be a 5 HT2 R stimulating or blocking agent, such as agomelatine or Saint John’s wort.

The Acid SphingoMyelinase enzyme (ASM, EC 3.1.4.12) is a lysosomal glycoprotein that catalyses the hydrolysis of sphingomyelin into ceramide and phosphorylcholine. Ceramide consecutively leads to membrane reorganization involving membrane rafts and downstream signalling that may result in apoptosis. In addition to ASM, at least three other sphingomyelinases have been described in mammalian cells that vary in their pH optimum, cofactor dependency and subcellular localization. Although these enzymes and an existing de novo synthesis pathway are alternative mechanisms for ceramide generation, activation of ASM is critical for at least some cellular responses, such as apoptosis induced by reactive nitrogen species, radiation, and CD95 ([5]). The activity of acid sphingomyelinase on the cell surface results in the formation of ceramide in the outer leaflet of the cell membrane. The generation of ceramide molecules within the outer leaflet alters the biophysical properties of the plasma membrane because the very hydrophobic ceramide molecules spontaneously associate with each other to form small ceramide-enriched membrane domains that fuse and form large, highly hydrophobic, tightly packed, gel-like ceramide-enriched membrane domains ([4]). As explained previously, the formation of these ceramide-enriched domains seems to be responsible of the entry of the SARS-CoV-2 virus in the epithelial cells.

The “agent of the invention” will be hereafter referred to as “FIASMA agent” or “agent having a FIASMA activity”, because it can effectively inhibit the ASM enzyme (the acid sphingomyelinase enzyme), directly or indirectly (the term “FIASMA” stands for Functional Inhibitor of Acid SphingoMyelinAse) and lead to decreased concentration of ceramide in the blood and in the cells. By extension, said term also encompass any agent that can lower the ceramide level at the surface of epithelial cells and in the blood by other means. In particular, it designates all agents that can in vitro lower the ASM activity and/or the ceramide surface level in epithelial cells by at least 50%, whatever the duration of incubation time. Determination of the ASM activity in these cells can be performed by conventional techniques, as disclosed in [4]. For example, ¹⁴C sphingomyelin can be used and contacted with Vero or Caco-2 cells in presence or absence of the agent of the invention. The release of ¹⁴C phosphorylcholine can then be monitored. Also, the Ceramide amount can be determined at the cell surface as disclosed in [4], e.g., by flow cytometry using anti-ceramide antibodies, or in the blood as measured by liquid chromatography coupled to tandem mass spectrometry (LC-MS/MS) consisting of an Acquity UPLC instrument (Waters Associates, Milford, MA, USA) coupled to a triple quadrupole (TQS Micro, Waters) mass spectrometer as detailed in Marin-Corral et al, 2021 ([57]).

Thus, as used herein, the terms “medication of the invention”, “treatment of the invention” or “agent of the invention” designate any compound that is able to efficiently inhibit the ASM enzyme (FIASMA agents) or to reduce the ceramide level at the surface of epithelial cells and thus to reduce the level of ceramides in the blood.

The agent of the invention can also reduce the activity of other sphingomyelinase enzymes, so as to reduce the levels of ceramide in epithelial cells and thus in the blood. Also, it can indirectly inhibit the activity of the sphingomyelinase enzymes by displacing them from lysosomal membranes, in particular intralysosomal vesicles, thereby releasing them into the lysosomal lumen and causing their partial degradation. Finally, it can also neutralize surficial ceramide by interacting with same or reducing the formation of same without inhibiting the ASM (for example diazepam). All these inhibitory activities ultimately lower the infection of epithelial cells with the SARS-CoV-2 virus.

This agent can be any agent whose FIASMA activity is demonstrated, including all the agents whose FIASMA activity has not been described yet, and will be discovered in the future.

The term “agent of the invention” also designates any derivative, analog, isomer, metabolite, salt, solvate, clathrate, polymorph, and co-crystal of the compounds mentioned below, provided that it has the inhibitory activities described above.

By “derivative”, it is meant herein a compound that is directly derived from a chemical compound of interest and is structurally similar though non-identical to said compound, and which retains the same biological activity and/or physico-chemical properties.

By “analog”, or “functional analog”, it is meant herein a compound that is not directly derived from a chemical compound of interest and is thus structurally different, but exhibits the same biological activity and/or physico-chemical properties, such as isosters.

By “isomer”, it is meant herein a compound having the same chemical formula as a compound of interest, but a different chemical structure. This term encompasses structural isomers and stereoisomers. Should the isomer of the invention be a stereoisomer, the individual stereoisomers (enantiomers and diastereoisomers) and mixtures thereof are included within the scope of the invention. Some of the compounds according to the invention may exist in tautomeric forms (a type of structural isomer), which are also included within the scope of the invention.

By “metabolite” as used herein, it is meant any compound that is an intermediate and/or a product of metabolism. A metabolite from a chemical compound is usually formed as part of the natural biochemical process of degrading and eliminating the compound of interest in a subject to which it is administered.

The term “solvate” according to the invention should be understood as meaning any form of the active agent in accordance with the invention (FIASMA agent), in which said compound is linked through non-covalent interactions to another molecule (normally a polar solvent), including especially hydrates and alcoholates, such as methanolate. Methods of solvation are well-known in the art.

By “clathrate”, it is meant herein a chemical substance consisting of a lattice or cage that entraps or contains a second type of molecule/compound of interest, and which can be used to increase the stability and solubility in water of the molecule/compound of interest. Clathrates are typically polymeric.

The term “polymorphs” means herein different crystalline forms of a compound of interest in which molecules have different arrangements and/or different molecular conformation. It includes crystalline liquid form or crystalline solid form of a compound of interest. Hydrates and clathrates can be polymorphs.

By “co-crystal”, it is meant herein a crystalline structure composed of at least two components, where the components may be atoms, ions or molecules. Solvates and clathrates may be co-crystals in certain conditions.

The term “pharmaceutically acceptable salt” is intended to mean, in the framework of the present invention, a salt of a compound which is pharmaceutically acceptable, as defined above, and which possesses the pharmacological activity of the corresponding compound.

The pharmaceutically acceptable salts comprise:
1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The agent of the invention is preferably contained in a pharmaceutical composition. This pharmaceutical composition, herein also called the “pharmaceutical composition of the invention”, therefore comprises the medication of the invention and a pharmaceutically acceptable carrier, which can be as defined above.

For the purpose of the invention, the term “pharmaceutically acceptable” is intended to mean what is useful to the preparation of a pharmaceutical composition, and what is generally safe and non-toxic, for a pharmaceutical use.

A “pharmaceutically acceptable carrier” or “excipient” refers to a non-toxic solid, semisolid or liquid filler, diluent, encapsulating material or formulation auxiliary of any type. In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

### Treatment of the COVID Disease

The present invention relates to the use of functional inhibitors of the acid sphingomyelinase enzyme (or FIASMA agents), for treating a viral infection due to at least one betacoronavirus in a patient in need thereof or to prepare a medicament intended to treat patients that have been diagnosed to be infected with at least one betacoronavirus. Said patient suffers for example from a symptomatic COVID-19 disease, more particularly from a strong or severe symptomatic COVID-19 disease. These agents can be used in particular for preventing and/or treating acute respiratory distress syndrome associated to said viral infection, or for lowering the risk of intubation or for increasing the survival rate of patients infected by said betacoronavirus.

### Treatment With Antidepressant Agents Having a FIASMA Activity

Several psychotropic medications, including certain antidepressants (i.e., amitriptyline, clomipramine, desipramine, doxepin, duloxetine, escitalopram, fluoxetine, fluvoxamine, imipramine, lofepramine, maprotiline, nortriptyline, paroxetine, protriptyline, sertraline, and trimipramine) have been shown to in vitro inhibit acid sphingomyelinase with varying degrees [4, 5, 12, 13]. Yet, in these studies, there are discrepancies whether citalopram, venlafaxine and mirtazapine do have in vitro FIASMA effect, which may be the case for longer incubation times. No clinical study to date has examined the potential usefulness of FIASMA psychotropic medications in patients hospitalized for COVID-19.

On the grounds of prior preclinical and clinical evidence suggesting that the acid sphingomyelinase (ASM)/ceramide system may provide a useful framework for better understanding SARS-CoV-2 infection and the repurposing of medications with functional inhibition of acid sphingomyelinase, called FIASMA medications, against COVID-19, it was hypothesized that FIASMA psychotropic medications could be significantly effective in reducing the risk of death or intubation among patients with mental disorder hospitalized for severe COVID-19. This population was focused on, because individuals with mental disorder are a vulnerable population at higher risk of severe COVID-19 [7, 17, 18] likely to receive psychotropic medications for treating or preventing the relapse of psychiatric disorders. No published study to date has examined the efficacy of any FIASMA psychotropic medication on COVID-19 in clinical populations hospitalized for severe COVID-19.

In a large observational multicenter retrospective study, the potential usefulness of FIASMA psychotropic medication use was examined among patients with mental disorder hospitalized for severe COVID-19. Study baseline was defined as the date of hospital admission for COVID-19 and the primary endpoint was a composite of intubation or death. This endpoint was compared between patients who received a psychotropic FIASMA medication and those who did not, in time-to-event analyses adjusted for sociodemographic characteristics, psychiatric and other medical comorbidity, and psychotropic and other medications. Patients without an end-point event had their data censored on May 1^{st}, 2020. The primary analysis was a Cox regression model with inverse probability weighting (IPW). Multiple sensitivity analyses were performed.

Of the 17,131 patients with a positive COVID-19 RT-PCR test who had been hospitalized for COVID-19, 1,963 (11.5%) were excluded because of missing data or their young age (i.e. less than 18 years old of age). Of 15,168 adult inpatients, 1,998 (13.2%) had a mental disorder diagnosis or an ongoing prescription of any antidepressant, antipsychotic, or mood stabilizer at hospital admission. Of these 1,998 patients, 827 (41.4%) had criteria for severe COVID-19. Of these 827 patients, 281 (34.0%) were excluded because they received a FIASMA psychotropic medication after 24 hours from hospital admission (N=277) or because they received an antipsychotic at baseline while being hospitalized in an ICU, possibly as an aid for intubation (N=5). Of the remaining 545 adult inpatients with mental disorder and severe COVID-19, 164 (30.1%) received a FIASMA psychotropic medication at baseline and 381 (69.9%) did not (Example 1, FIGS. 1 and 2).

In this multicenter retrospective observational study involving a relatively large sample of patients with mental disorder hospitalized for severe COVID-19 (N=545), it was found that FIASMA psychotropic medication use at study baseline was significantly with a 50% reduction in risk of intubation or death, independently of sociodemographic characteristics, psychiatric and other medical comorbidity, and psychotropic and other medications. This association remained significant in multiple sensitivity analyses. Secondary exploratory analyses suggested that this association was not specific to one FIASMA psychotropic class or medication in this population.

In a preferred embodiment, the present invention relates to the use of antidepressant agents having FIASMA activity, preferably 5 HT R-stimulating or blocking agents (in particular 5 HT1R-stimulating or blocking agents or 5HT2R-stimulating or blocking agents) having FIASMA activity, to prepare a medicament intended to treat patients that have been diagnosed to be infected with at least one betacoronavirus.

The present invention also discloses a method for preventing and/or treating any subject that has been diagnosed to be infected with at least one betacoronavirus, said method comprising administering to said subject an effective amount of an antidepressant agent having FIASMA activity, for example a 5 HTR-stimulating or blocking agent (in particular 5 HT1R-stimulating or blocking agents or 5HT2R-stimulating or blocking agents) having FIASMA activity. The method of the invention can comprise a diagnosis step as preliminary step. The said infection can be diagnosed or detected by any conventional means, such as molecular biological detection and/or viral titration of respiratory specimens, or by assaying blood for antibodies specific for said betacoronavirus.

In a particular embodiment, the antidepressant agent of the invention, having FIASMA activity, is used for preventing and/or treating patients infected by a Severe Acute Respiratory Syndrome-related coronavirus (SARS-CoV), in particular SARS-CoV-2.

In another particular embodiment, the agents of the invention are intended to treat patients suffering from a symptomatic COVID-19 disease, and more particularly from a strong or severe symptomatic COVID-19 disease, as defined above. In a particular embodiment, the treatment of the invention is administered to subjects (animals or humans) that experience for example difficulty in breathing and/or a lack of oxygen.

In another particular embodiment of the invention, the antidepressant agents of the invention are capable of preventing and/or treating acute respiratory distress syndrome associated to said viral infection, of lowering the risk of intubation and / or of increasing the survival rate of patients infected by said betacoronavirus, as described in the examples below.

In a preferred embodiment, said subject is a human patient. Said patient can be older than 75 years old, between 65 and 74 years old, between 18 and 65 years old, or below 18 years old. In a more preferred embodiment, said human patient is older than 50 years old, even more preferably older than 70 years old. These people indeed usually have greater mortality rates than younger people when infected with said virus.

In a preferred embodiment, said subject is an animal chosen in the group consisting of: bats, hedgehog, camels, mice, humans, pigs, cats, among others. Any other animal that can be infected with a betacoronavirus could also benefit from the treatment of the invention.

The pharmaceutical composition of the invention can contain any antidepressant agent having FIASMA activity that can lower the risk of intubation and/or death of a COVID patient. In particular, said agent can be a 5-HT R-stimulating or blocking agent having FIASMA activity, including escitalopram, citalopram, sertraline, fluoxetine, paroxetine, mirtazapine, venlafaxine, duloxetine, or clomipramine ([4]).

In a particular embodiment, said antidepressant agent having FIASMA activity is selected in the group consisting of: Amitriptyline, Sertraline, Protriptyline, Fluoxetine, Nortriptyline, Maprotiline, Trimipramine, Desipramine, Lofepramine, Clomipramine, Duloxetine, Paroxetine, Imipramine, Fluvoxamine, Doxepin, Escitalopram, Citalopram, venlafaxine and mirtazapine.

In a more particular embodiment, said antidepressant agent having FIASMA activity is selected in the group consisting of: Amitriptyline, Sertraline, Protriptyline, Fluoxetine, Nortriptyline, Maprotiline, Trimipramine, Desipramine, Lofepramine, Clomipramine, Duloxetine, Paroxetine, Imipramine, Fluvoxamine, Doxepin, Escitalopram and Citalopram.

In a more particular embodiment, said antidepressant agent having FIASMA activity is selected in the group consisting of: Sertraline, Fluoxetine, Clomipramine, Duloxetine, Paroxetine, Fluvoxamine, Escitalopram, Citalopram, venlafaxine and mirtazapine.

In another preferred embodiment, the antidepressant agent used in the composition of the invention is chosen in the group consisting of: escitalopram, venlafaxine, citalopram, sertraline, fluoxetine, paroxetine, duloxetine, mirtazapine, or clomipramine. In an even more preferred embodiment, the antidepressant agent used in the composition of the invention is chosen in the group consisting of: escitalopram, venlafaxine, paroxetine, fluoxetine, and mirtazapine.

In another preferred embodiment, the antidepressant used in the composition of the invention is chosen in the group consisting of: Dosulepin, Vortioxetine, and Milnacipran.

In another preferred embodiment, the antidepressant used in the composition of the invention is Diazepam.

In a more preferred embodiment, the antidepressant agent used in the composition of the invention is a SSRI having a FIASMA activity, such as Fluoxetine or Fluvoxamine.

The composition of the invention can contain a combination of at least two antidepressant agents having FIASMA activity as defined above. The total FIASMA dose received, calculated as the sum of the doses of each FIASMA medication that is received by the patient, multiplicated by the degree of in vitro inhibition of ASM of each of these FIASMA medications, is hypothesized to be correlated with mortality reduction in COVID-19. Therefore, the higher this total dose is, the lower the mortality will be.

It is noteworthy that the daily dose of the antidepressant agent(s) to be administered to the infected subject is preferably the conventional dose used in the art for antidepressant purposes. Importantly, this could not have been predicted from any prior art study, even from those suggesting that some antidepressant molecules may have an effect on the endocytic virus entry. The present invention shows for the first time that using conventional non-toxic doses (no need of higher doses) is enough to efficiently impair virus infection and reduce the mortality of the COVID disease.

This dose is for example comprised between about 5 mg/day and about 60 mg/day for fluoxetine-equivalent dose, said dose being as defined in [50]. Preferably, low to moderate doses of 10 mg/day to 40 mg/day are used in the context of the invention among adults aged more than 70 years old of age, so as to minimize side effects. As a matter of fact, low and moderate doses of antidepressants are generally well tolerated, especially when they are used on a short period, including in older adults who are the most prone to develop severe Covid-19 infection.

According to a preferred embodiment, said antidepressant agent can be administered to the subject at an effective Fluoxetine-equivalent dose comprised between about 20 mg/day and about 60 mg/day, preferably between about 30 mg/day and about 60 mg/ day, more preferably between about 40 mg/day and about 60 mg/day for parenteral or oral administration.

For example, if Fluoxetine is used, the dose to be administered can be comprised between about 20 mg/day and about 60 mg/day, preferably between about 30 mg/day and about 60 mg/ day, more preferably between about 40 mg/day and about 60 mg/day for parenteral or oral administration.

If Fluvoxamine is used, the dose to be administered can be comprised between about 150 mg/day and about 300 mg/day, preferably between about 200 mg/day and about 300 mg/ day, more preferably between about 250 mg/day and about 300 mg/day for parenteral or oral administration.

In other words, the dose of antidepressant contained in the composition of the invention is such that, when converted into dose-equivalent to fluoxetine-equivalent dose as explained in Hayasaka Y ([50]), it is comprised in the above-mentioned ranges.

According to this study, the following doses of other antidepressant agents are equivalent to 40 mg/day of Fluoxetine:
paroxetine : 34.0 mg/day,
clomipramine : 116.1 mg/day,
desipramine : 196.3 mg/day,
escitalopram : 18.0 mg/day,
fluvoxamine : 143.3 mg/day,
mirtazapine 50.9 mg/day,
sertraline 98.5 mg/day,
venlafaxine 149.4 mg/day.

These doses are thus recommended as highest dose in the treatment of the invention.

Yet, according to another preferred embodiment, said antidepressant agent can be administered to the subject at an effective Fluoxetine-equivalent dose orally or by intravenous perfusion comprised between 5 mg and 60 mg per day, and preferably between 10 mg and 50 mg in older adults aged 70 years and over, and between 30 mg and 60 mg in younger adults aged 18 to 69 years old of age.

It is within the skill of the person in the art to determine the desired therapeutic amount of antidepressant agent to deliver by routine methods in the art.

The pharmaceutical composition of the invention may preferably be in a form suitable for the purposes of the invention. For example, said composition may be in a form suitable for parenteral or oral administration, such as a liquid suspension, or a solid dosage form (granules, pills, capsules or tablets). The term “parenteral” as used herein includes subcutaneous injection, intravenous, or intramuscular, injection.

In a preferred embodiment, the agent of the invention is administered to the patient once it is diagnosed from severe COVID disease, and until the patient displays at least one symptom of the COVID disease. The treatment preferably ceases when the infection is over. Such short-term treatments are well tolerated and do not induce any side-effect or dependency in the treated patients.

### Treatment With FIASMA Non-Psychotropic Medications

Several other medications, including antipsychotic agents (Triflupromazine, Trifluoperazine, Thioridazin, Sertindole, Fluphenazine, Flupenthixol, Perphenazine, Aripiprazole, Penfluridol, Chlorprothixene, Pimozide, Promazine, Chlorpromazine), medications of the nervous system (cinnarizine, flunarizine), certain antihistaminic medications (astemizole, clemastine, cyproheptadine, desloratadine, loratadine, mebhydrolin, pimethixene, promethazine, terfenadine), certain anticholinergic antiparkinson medications (benztropine, biperidene, profenamine), antiprotozoal medications (emetine, quinacrine), calcium channel blockers (amlodipine, bepridil, fendiline, mibefradil, perhexiline), beta blocking agents (carvedilol), antiarrhythmics (amiodarone, aprindine), medications for functional gastointestinal disorders (alverine, camylofin, dicycloverine, mebeverine), antivertigo medications (cinnarizine, flunarizine), and natural products (conessine, solasodine, tomatidine), vasodilators (dilazep, suloctidil), cough suppressants (cloperastine), antidiarrheal medications (loperamide), antimycobacterials (clofazimine), endocrine therapy medications (tamoxifen), or muscle relaxants (cyclobenzaprine) have also shown to in vitro inhibit acid sphingomyelinase with varying degrees [4, 5, 12, 13]. No clinical study to date has examined the potential usefulness of any of these FIASMA medications in patients hospitalized for COVID-19.

In a large observational multicenter retrospective study, the potential usefulness of FIASMA (non-psychotropic) medication use was examined among patients hospitalized for severe COVID-19. Study baseline was defined as the date of hospital admission for COVID-19 and the primary endpoint was a composite of intubation or death. This endpoint was compared between patients who received a (non-psychotropic) FIASMA medication (e.g., loperamide, amlodipine, amiodarone, aripiprazole, chlorpromazine, and desloratadine) and those who did not, in time-to-event analyses adjusted for sociodemographic characteristics and medical comorbidity. It was observed that FIASMA medication use at baseline was significantly associated with a 42% reduction of risk of intubation or death among adult patients hospitalized for severe COVID-19.

More precisely, it was found that all tested non-psychotropic FIASMA medications (loperamide, amlodipine, amiodarone, aripiprazole, chlorpromazine, and desloratadine), prescribed at usual doses for other indications, were effective preventive and curative treatments for reducing risk of death or intubation in adult patients with severe COVID-19.

More precisely, as shown in example 3, the present inventors performed a large (7,345 adult inpatients with COVID-19, including 138 (1.9%) patients receiving hydroxyzine during the hospitalization) multicenter observational retrospective study, and examined the association between hydroxyzine use with the risk of death, ascertained by death certificates, among male and female adult patients (aged 18 to 103 years old of age) who have been admitted to these medical centers with COVID-19, ascertained by a positive reverse-transcriptase-polymerase-chain-reaction (RT-PCR) test from analysis of nasopharyngeal or oropharyngeal swab specimens.

They found that hydroxyzine, prescribed for others indications (including urticaria, allergic rhinitis, hay fever, conjunctivitis, pruritis) and for its tranquilizer and sedative properties, are significantly and substantially associated with reduced risk of death in hospitalized adult patients with Covid-19, independently of patients’ characteristics, disease’s severity and use of other psychotropic medications (FIG. 6).

In another embodiment, the present invention therefore relates to the use of non-psychotropic agents having FIASMA activity, to prepare a medicament intended to treat patients that have been diagnosed to be infected with at least one betacoronavirus.

These non-psychotropic FIASMA agents can be, for example:
antipsychotic agents such as Triflupromazine, Trifluoperazine, Thioridazin, Sertindole, Fluphenazine, Flupenthixol, Perphenazine, Aripiprazole, Penfluridol Chlorprothixene, Pimozide, Promazine, Chlorpromazine,
medications of the nervous system such as cinnarizine and flunarizine,
certain antihistaminic medications such as astemizole, clemastine, cyproheptadine, desloratadine, loratadine, mebhydrolin, pimethixene, promethazine, and terfenadine,
certain anticholinergic antiparkinson medications such as benztropine, biperidene, profenamine,
antiprotozoal medications such as emetine and quinacrine,
calcium channel blockers such as amlodipine, bepridil, fendiline, mibefradil, and perhexiline,
beta blocking agents such as carvedilol,
antiarrhythmics such as amiodarone and aprindine,
medications for functional gastointestinal disorders such as alverine, camylofin, dicycloverine, Dicyclomine and mebeverine,
mucolytic agent such as ambroxol,
antivertigo medications such as cinnarizine and flunarizine,
natural products such as conessine, solasodine, and tomatidine
vasodilators such as dilazep and suloctidil,
cough suppressants such as cloperastine,
antidiarrheal medications such as loperamide,
antimycobacterials such as clofazimine,
endocrine therapy medications such as tamoxifen,
Sex hormone such as Clomifene, or
muscle relaxants such as cyclobenzaprine.

All these compounds have been shown to exhibit a FIASMA activity as meant in the present invention, as they can inhibit acid sphingomyelinase in vitro with varying degrees [4, 5, 12, 13].

The composition of the invention can contain a combination of at least two non-psychotropic agents or a combination of psychotropic and non-psychotropic agents having FIASMA activity as defined above. It is preferred to combine at least one psychotropic agent and at least one non-psychotropic agent, among those defined above.

The total FIASMA dose received, calculated as the sum of the doses of each FIASMA medication that is received by the patient, multiplicated by the degree of in vitro inhibition of ASM of each of these FIASMA medications, is hypothesized to be correlated with mortality reduction in COVID-19. Therefore, the higher this total dose is, the lower the mortality will be.

The present invention therefore proposes to use any of these non-psychotropic FIASMA agents for preventing severe forms of the COVID 19 disease to occur, or to treat patients that have been diagnosed to be infected with at least one betacoronavirus. The use of any of these compounds or any combination of these compounds is encompassed within the present invention.

In a particular embodiment, the present invention proposes to use antipsychotic agents such as Triflupromazine, Trifluoperazine, Thioridazin, Sertindole, Fluphenazine, Flupenthixol, Perphenazine, Aripiprazole, Penfluridol Chlorprothixene, Pimozide, Promazine, Chlorpromazine for preventing or treating a viral infection due to at least one betacoronavirus such as SARS-CoV-2, in a patient in need thereof. Said patient suffers for example from a symptomatic COVID-19 disease, more particularly from a strong or severe symptomatic COVID-19 disease. These agents can be used in particular for preventing and/or treating acute respiratory distress syndrome associated to said viral infection, or for lowering the risk of intubation or for increasing the survival rate of patients infected by said betacoronavirus.

In another particular embodiment, the present invention proposes to use any other non psychotropic FIASMA agents as disclosed above, for example cinnarizine, flunarizine, benztropine, biperidene, profenamine, emetine, quinacrine, amlodipine, bepridil, fendiline, mibefradil, perhexiline, carvedilol, amiodarone, aprindine, alverine, camylofin, dicycloverine, mebeverine, cinnarizine, flunarizine, conessine, solasodine, tomatidine, dilazep, suloctidil, cloperastine, loperamide, clofazimine, tamoxifen, or cyclobenzaprine, for preventing or treating a viral infection due to at least one betacoronavirus such as SARS-CoV-2, in a patient in need thereof. Said patient suffers for example from a symptomatic COVID-19 disease, more particularly from a strong or severe symptomatic COVID-19 disease.

Other compounds having FIASMA activity, such as ambroxol or natural products such as Tomatidine, Conessine or Solasodine, can also be used in this respect.

In a particular embodiment, the present invention proposes to use a non-psychotropic FIASMA agent chosen in the group consisting of: Triflupromazine, Trifluoperazine, Thioridazin, Sertindole, Fluphenazine, Flupenthixol, Perphenazine, Aripiprazole, Penfluridol Chlorprothixene, Pimozide, Promazine, Chlorpromazine, cinnarizine, flunarizine, benztropine, biperidene, profenamine, emetine, quinacrine, amlodipine, bepridil, fendiline, mibefradil, perhexiline, carvedilol, amiodarone, aprindine, alverine, camylofin, dicycloverine, mebeverine, cinnarizine, flunarizine, conessine, solasodine, tomatidine, dilazep, suloctidil, cloperastine, loperamide, clofazimine, tamoxifen, and cyclobenzaprine.

In a particular embodiment, the present invention proposes to use a non-psychotropic FIASMA agent chosen in the group consisting of: astemizole, clemastine, cyproheptadine, desloratadine, loratadine, promethazine, and terfenadine. In another particular embodiment, the present invention proposes to use mebhydrolin, or pimethixene.

In a more particular embodiment, the present invention proposes to use loperamide, amlodipine, amiodarone, aripiprazole, chlorpromazine, or desroratadine for preventing or treating a viral infection due to at least one betacoronavirus such as SARS-CoV-2, in a patient in need thereof. Said patient suffers for example from a symptomatic COVID-19 disease, more particularly from a strong or severe symptomatic COVID-19 disease.

All the above mentioned non-psychotropic agents can be used in particular for preventing and/or treating acute respiratory distress syndrome associated to said viral infection, or for lowering the risk of intubation or for increasing the survival rate of patients infected by said betacoronavirus. In another particular embodiment of the invention, these agents are capable of preventing and/or treating acute respiratory distress syndrome associated to said viral infection, of lowering the risk of intubation and / or of increasing the survival rate of patients infected by said betacoronavirus, as described in the examples below.

In the context of the present invention, these non-psychotropic FIASMA medications can be prescribed at their usual doses for their other indications (cf. Vidal for example).

In a preferred embodiment, said subject is a human patient. Said patient can be older than 75 years old, between 65 and 74 years old, between 18 and 65 years old, or below 18 years old. In a more preferred embodiment, said human patient is older than 50 years old, even more preferably older than 70 years old. These people indeed usually have greater mortality rates than younger people when infected with said virus.

In a preferred embodiment, said subject is an animal chosen in the group consisting of: bats, hedgehog, camels, mice, humans, pigs, cats, among others. Any other animal that can be infected with a betacoronavirus could also benefit from the treatment of the invention.

The pharmaceutical composition of the invention containing these non-psychotropic FIASMA agents may preferably be in a form that is suitable for the purposes of the invention. For example, said composition may be in a form suitable for parenteral or oral administration, such as a liquid suspension, or a solid dosage form (granules, pills, capsules or tablets). The term “parenteral” as used herein includes subcutaneous injection, intravenous, or intramuscular, injection.

In this aspect, the present invention relates to non-psychotropic FIASMA agents for use for treating a viral infection due to a Severe Acute Respiratory Syndrome-related coronavirus (SARS-CoV), in particular SARS-CoV-2, in a patient in need thereof. It also relates to said non-psychotropic FIASMA agent, for its use for treating patients suffering from a symptomatic COVID-19 disease, advantageously from a strong or severe symptomatic COVID-19 disease. It also relates to said non-psychotropic FIASMA agent, for its use for preventing and/or treating acute respiratory distress syndrome associated to said viral infection. Finally, it also relates to said non-psychotropic FIASMA agent, for its use for lowering the risk of intubation and/or for lowering the risk of hospitalization and/or for increasing the survival rate of patients (i.e., decreasing risk of death) infected by said coronavirus.

In other words, the present invention relates to the use of a non-psychotropic FIASMA agent as described above, for the preparation of a medicament that is intended to treat a symptomatic COVID-19 disease, advantageously from a strong or severe symptomatic COVID-19 disease; or an acute respiratory distress syndrome associated to said viral infection.

The present invention also discloses a method for treating a viral infection due to a Severe Acute Respiratory Syndrome-related coronavirus (SARS-CoV), in particular SARS-CoV-2, in a patient in need thereof, said method comprising administering in said patient a significant amount of non-psychotropic FIASMA agents. It also discloses a method for treating patients suffering from a symptomatic COVID-19 disease, a method for preventing and/or treating acute respiratory distress syndrome associated to said viral infection, and a method for lowering the risk of intubation and/or for increasing the survival rate (i.e., decreasing risk of death) of patients infected by said coronavirus, said methods comprising administering in said patients a significant amount of non-psychotropic FIASMA agents.

It is noteworthy that the daily dose of the non-psychotropic agent(s) to be administered to the infected subject is preferably the conventional non-toxic dose used in the art for the indications for which they were accepted by FDA or EMEA. Importantly, this could not have been predicted from any prior art study. The present invention shows for the first time that using conventional non-toxic doses of these agents (no need of higher doses) is enough to efficiently impair virus infection and reduce the mortality of the COVID disease.

Therefore, in the context of the treating method of the invention, the non-psychotropic agents can be administered at their conventional doses for which they have been approved by at least one competent national or regional health authority (e.g., FDA or EMEA).

The non-psychotropic FIASMA agent of the invention is preferably incorporated in a pharmaceutical composition, said composition containing, apart from said agent, a pharmaceutically acceptable excipient as defined above.

The pharmaceutical composition of the invention may preferably be in a form suitable for the purposes of the invention. For example, said composition may be in a form suitable for parenteral or oral administration, such as a liquid suspension, or a solid dosage form (granules, pills, capsules or tablets). The term “parenteral” as used herein includes subcutaneous injection, intravenous, or intramuscular, injection. The pharmaceutical composition of the invention is more preferably administered orally.

In a preferred embodiment, the non-psychotropic agent of the invention is administered to the patient once it is diagnosed from severe COVID disease, and until the patient displays at least one symptom of the COVID disease. The treatment preferably ceases when the infection is over. Such short-term treatments are well tolerated and do not induce any side-effect or dependency in the treated patients.

In a particular embodiment, the non-psychotropic agent of the invention is an inhibitor of the histamine H1-receptor (hereafter “inhibitor of the invention” or “H1 inhibitor”).

More generally, the present invention proposes to use inhibitors of peripheral Histamine receptors having FIASMA activity for treating a viral infection due to a Severe Acute Respiratory Syndrome-related coronavirus (SARS-CoV), in particular SARS-CoV-2, in a patient in need thereof. Said inhibitors can also be efficient for treating patients suffering from a symptomatic COVID-19 disease, advantageously from a strong or severe symptomatic COVID-19 disease. Particularly, said inhibitors can prevent and/or treat acute respiratory distress syndrome associated to said viral infection ; and/or lower the risk of intubation and/or lower the risk of hospitalization and/or eventually increase the survival rate of patients infected by said coronavirus.

There are four histamine receptors which bind histamine as their primary endogenous ligand (namely the H1 receptor, H2 receptor, H3 receptor, and H4 receptor). Although the inventors have gathered data involving anti-H1 compounds, the three other histamine receptors are likely to be involved in the regulation of the COVID-related mortality as well, because of the well-known cross regulation occurring between all these histamine receptors, provided that they also have a FIASMA activity.

In a particular embodiment, the present invention relates to the use of inhibitors of peripheral H2 receptors, of peripheral H3 receptors, or of peripheral H4 receptors, wherein said inhibitors have a FIASMA activity. These inhibitors are preferably chosen in the group consisting of: astemizole, clemastine, cyproheptadine, desloratadine, loratadine, mebhydrolin, pimethixene, promethazine, hydroxyzine and terfenadine, and preferably astemizole, clemastine, cyproheptadine, desloratadine, loratadine, promethazine, hydroxyzine and terfenadine, or a pharmaceutical salt or solvate thereof. Stereoisomers and conformers thereof are also herein encompassed.

The present invention more precisely relates to the use of any of these anti-histaminic inhibitors for treating a viral infection due to a Severe Acute Respiratory Syndrome-related coronavirus (SARS-CoV), in particular SARS-CoV-2, in a patient in need thereof. Said inhibitors can also be efficient for treating patients suffering from a symptomatic COVID-19 disease, advantageously from a strong or severe symptomatic COVID-19 disease. Particularly, said inhibitors can prevent and/or treat acute respiratory distress syndrome associated to said viral infection; and/or lower the risk of intubation and/or lower the risk of hospitalization and/or eventually increase the survival rate of patients infected by said coronavirus.

The histamine H1-receptor is a member of the superfamily of G-protein-coupled receptors (GPCRs). Histamine cross links sites on transmembrane domains III and V to stabilize the receptor in its active conformation, thus causing the equilibrium to switch to the position “on”. H1 antihistamine, which are not structurally related to histamine, do not antagonize the binding of histamine but bind to different sites on the receptor, to produce the opposite effect. Thus, H1-antihistamines are not receptor antagonists, but inverse agonists of the H1 receptor.

In some embodiments, the H1 inhibitor of the invention inhibits the activity of the H1 receptor by at least 5%, 10%, 20%, 30%, 40%, 50%, 60%, 70%, 80%, 90%, 95% or 100% (i.e., complete loss of activity) relative to a control. In some embodiment, the inhibitor of the invention is capable of binding to an allosteric site of the histamine H1 receptor. In a particular embodiment, the inhibitor of the invention has an IC₅₀ of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, less than or equal to 0.001 nM. Said inhibitory effect can be tested by conventional means, such as those disclosed in Diaz Nebreda et al ([58]).

In a preferred embodiment, the H1 inhibitor of the invention is hydroxyzine or a pharmaceutical salt or solvate thereof. Stereoisomers and conformers thereof are also herein encompassed.

Hydroxyzine is a member of the diphenylmethylpiperazine class of antihistamines. It is sold under the brand names Atarax and many others (Vistaril, Equipose, Masmoran, Paxistil, Alamon, Aterax, Durrax, Tran-Q, Orgatrax, Quiess, Tranquizine, etc.). Its CAS number is 68-88-2. It is also known as hydroxizine, or hydroxycine. Its chemical formula is 2-[2-[4-[(4-chlorophenyl)-phenylmethyl]piperazin-1-yl]ethoxy]ethanol:

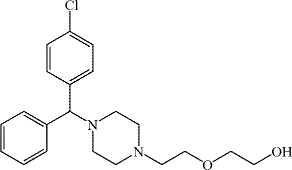 [SMILES: OCCOCCN1CCN(C(c2ccccc2)c2ccc(Cl)cc2)CC1]

Any salt thereof can be used in the invention. Preferably, said salt can be a pamoate (CAS 10246-75-0) or a hydrochloride or a di-hydrochloride salt (CAS2192-693-1). These salts are commercially available and therefore very easy to acquire and use: Vistaril, Equipose, Masmoran, and Paxistil are preparations of the pamoate salt, while Atarax, Alamon, Aterax, Durrax, Tran-Q, Orgatrax, Quiess, and Tranquizine are of the hydrochloride salt.

Among first generation antihistamines, hydroxyzine is one of the most prescribed in France. Beyond its antihistamine activity, hydroxyzine is also prescribed as a psychotropic medication for its tranquilizer and sedative properties, as it weakly acts as an antagonist of the serotonin 5-HT2A-receptor, the dopamine D2 receptor, and the α1-adrenergic receptor. Because prior research supports that severe COVID-19 is characterized by an overexuberant inflammatory response and that viral load has been associated with the worsening of symptoms it was hypothesized that the antihistamine compound hydroxyzine could be potentially effective in reducing the risk of death in patients with COVID-19. The results disclosed below (example 3) show that hydroxyzine, prescribed for others indications (including urticaria, allergic rhinitis, hay fever, conjunctivitis, pruritis) and for its tranquilizer and sedative properties, are significantly and substantially associated with reduced risk of death in hospitalized adult patients with Covid-19, independently of patients’ characteristics, disease’s severity and use of other psychotropic medications.

In the context of the invention, hydroxyzine will be preferably administered orally or via intramuscular injection.

As mentioned above, the daily dose of the H1 inhibitors of the invention to be administered to the infected subject is preferably the conventional dose used in the art and approved for said H1 antihistamine compounds. This dose is for example comprised between about 12.5 mg/day and about 400 mg/day for hydroxyzine dose, when hydroxyzine is administered orally or through intramuscular injection.

In a preferred embodiment of the invention, the H1 inhibitor of the invention is hydroxyzine which is administered orally at an effective dose comprised between about 12.5 mg/day and about 400 mg/day, preferably between about 25 mg/day and about 200 mg/day, preferably between about 50 mg/day and about 100 mg/day.

Preferably, low to moderate doses are used in the context of the invention, so as to minimize side effects of the antihistamine compound. As a matter of fact, low and moderate doses of antihistamine compound are generally well tolerated, especially when they are used on a short period, including in older adults who are the most prone to develop severe Covid-19 infection.

According to a preferred embodiment, the H1 inhibitors of the invention in a general manner are administered to the subject at a dose comprised between about 1 mg/day and about 400 mg/day, preferably between about 5 mg/day and about 300 mg/day, preferably 12.5 mg/day and about 400 mg/day, preferably between about 25 mg/day and about 300 mg/day, more preferably between about 50 mg/day and about 200 mg/day and most preferably between about 50 mg/day and about 100 mg/day (in particular if said inhibitor is administered orally).

According to another preferred embodiment, the H1 inhibitors of the invention are administered to the subject orally or by intramuscular perfusion at an effective dose comprised between about 1 mg/day and about 400 mg/day, preferably between about 5 mg/day and about 300 mg/day, preferably between about 12.5 mg and 400 mg per day, and preferably between 12.5 mg and 100 mg in older adults aged 70 years and over, and between 50 mg and 400 mg in younger adults aged 18 to 69 years old of age. These doses have to be divided by 2 to 4 folds if the patient furthermore suffers from renal impairment (<50 mL/min) or liver failure.

It is within the skill of the person in the art to determine the desired therapeutic amount of the inhibitor of the invention to deliver by routine methods in the art.

### Personalised Treatment of the COVID Disease and Prognosis of the Disease

In a particular embodiment of the invention, the amount of the medication of the invention is determined by quantifying the level of circulating ceramides in a biological sample of the patient, prior to the treatment, or after initiating the treatment in order to adapt the dose of the treatment, in a personalized-medicine perspective.

Ceramides are bioactive lipids involved in inflammation, apoptosis, obesity, and insulin resistance, and are biomarkers of cardiovascular diseases. Circulating ceramides are for example long chain ceramides such as C18:0, C16:0 and C24:1 or hexosylceramides.

As explained above, the ceramide level can be determined at the cell surface of blood cells, e.g., by flow cytometry using anti-ceramide antibodies, or by liquid chromatography coupled to tandem mass spectrometry (LC-MS/MS) on plasma or serum samples.

Thus, the treating method of the invention may also contain a first step of detecting the level of ceramide in a biological sample of the patient by appropriate means. Said biological sample is preferably a blood, plasma or serum sample obtained from the patient.

If the total ceramide plasma level is low in the patient sample (i.e., when this level is as low as in healthy subjects, i.e. <500 µmol/l), then the virus will not easily enter into the target cells and the patient may have a good prognosis, with or without treatment. It can therefore be said that the lower the total circulating ceramide level is, the more benign the COVID disease will be (provided that the ceramide level remains at such a low level). For example, the COVID disease shall remain asymptomatic or at a mild stage, at defined above.

By contrast, when the ceramide level is high (e.g., higher than the mean ceramide level measured in the blood of healthy subjects, i.e., has become superior to 500 µmol/l), then the prognosis of the disease is bad and the patient is at risk of experiencing a strong form of the COVID disease. This should be prevented by initiating a treatment of the invention or increasing the dosage regimen of this treatment, or by administering two or more FIASMA agents concomitantly, for example if the maximum allowed dose of the treatment is already prescribed. The efficiency of the treatment(s) can then be assessed by controlling the level of total circulating ceramide in the blood samples of the patient, at different time points after the treatment has been administered, until it has sufficiently decreased (i.e., has reached a level <500 µmol/l).

The level of circulating long ceramides such as C18:0, C16:0 and C24:1 can therefore advantageously serve as a prognostic marker, for example in a prognosis score, alone or in combination with other parameters (e.g., the total ceramide plasma level, the plasma levels of C18:0, C16:0 and C24:1, the ratio of hexosylceramides/ceramides, etc.) to assess the severity of the COVID disease and help optimize the treatment in terms of dose and number of treatments.

This has been confirmed recently by another team, especially for C18:0, C16:0 and C24:1 [57].

This marker can also be helpful to screen the efficacity of new medications, whose FIASMA activity is yet unknown, for their ability to prevent or treat strong forms of the COVID disease.

### Treatment of Other Diseases Involving ASM

As disclosed above, FIASMA medications include a wide range of medications from different pharmaceutical classes. The present results can suggest that any of these FIASMA medications may be useful in other diseases associated with observed increased acid sphingomyelinase activity thanks to its anti-apoptotic and neuroprotective effects.

These other diseases include (cf. [13]): brain ischemia, stroke, ethanol-induced neuronal cell death, Alzheimer’s dementia, Parkinson’s disease, Wilson’s disease, Chorea Huntington, spinal cord injury, seizure disorder, glaucoma, major depression, inflammatory bowel disease, acute and chronic lung injury, and other infections associated with increased acid sphingomyelinase activity such as endotoxic shock syndrome, severe sepsis, infection by Neisseria gonorrhoeae.

FIASMA agents could also be used to protect against neurodegeneration occurring in multiple sclerosis, the radiation and chemotherapy-induced lethal gastrointestinal syndrome, and morphine anti-nociceptive tolerance.

Therefore, in another aspect, the present invention relates to the use of the FIASMA agents of the invention, for preventing and/or treating any of the following diseases: brain ischemia, stroke, ethanol-induced neuronal cell death, Alzheimer’s dementia, Parkinson’s disease, Wilson’s disease, Chorea Huntington, spinal cord injury, seizure disorder, glaucoma, major depression, inflammatory bowel disease, acute and chronic lung injury, and other infections associated with increased acid sphingomyelinase activity such as endotoxic shock syndrome, severe sepsis, infection by Neisseria gonorrhoeae or Ebola virus, multiple sclerosis, radiation and chemotherapy-induced lethal gastrointestinal syndrome, and morphine anti-nociceptive tolerance.

Methods for treating any of these diseases, involving the administration of the FIASMA medications of the invention in patients in need thereof, are also encompassed within the present invention.

These FIASMA medications can be psychotropic or non-psychotropic. They are for example antidepressants, antipsychotics, anti-histaminiques agents having FIASMA activity, as detailed above. Other compounds having FIASMA activity, for example natural products such as Tomatidine, Conessine or Solasodine, can also be used in this respect.

It is noteworthy that, in this aspect of the invention, any FIASMA agent as described above could be used to improve the prognosis of any of the following diseases: brain ischemia, stroke, ethanol-induced neuronal cell death, Alzheimer’s dementia, Parkinson’s disease, Wilson’s disease, Chorea Huntington, spinal cord injury, seizure disorder, glaucoma, major depression, inflammatory bowel disease, acute and chronic lung injury, and other infections associated with increased acid sphingomyelinase activity such as endotoxic shock syndrome, severe sepsis, infection by Neisseria gonorrhoeae or Ebola virus, multiple sclerosis, radiation and chemotherapy-induced lethal gastrointestinal syndrome, and morphine anti-nociceptive tolerance.

The therapeutic effect of these compounds on these diseases has never been experimentally validated before.

### FIGURE LEGENDS

FIG. 1 describes the characteristics of the studied cohort of EXAMPLE 1.

FIG. 2 shows the Kaplan-Meier curves of the data exposed in EXAMPLE 1, for the composite endpoint of intubation or death in the full sample crude analysis (N=545) (A), in the full sample analysis with IPW (N=545) (B), and in the matched analytic sample using a 1:1 ratio (N=328) (C) among patients with mental disorder hospitalized for severe COVID-19, according to FIASMA psychotropic medication use at baseline. The shaded areas represent pointwise 95% confidence intervals. Risk tables are displayed for the full sample crude analysis in A, for the full sample with the inverse-probability-weighting weights in B and for the matched analytic sample using a 1:1 ratio in C.

FIG. 3 describes the characteristics of the studied cohort of EXAMPLE 2.

FIG. 4 discloses the Kaplan-Meier curves obtained in EXAMPLE 2 for the composite endpoint of intubation or death in the full sample crude analysis (N=2846) (A), in the full sample analysis with IPW (N=2846) (B), and in the matched analytic sample using a 1:1 ratio (N=554) (C) among patients hospitalized for severe COVID-19, according to FIASMA medication use at baseline. The shaded areas represent pointwise 95% confidence intervals.

FIG. 5 describes the main characteristics of the Study cohort.

FIG. 6 shows kaplan-Meier curves for death in the full sample (A) (N=7,345) and in the matched analytic sample (B) (N=276) of patients who had been admitted to the hospital for Covid-19 according to hydroxyzine use. The shaded areas represent pointwise 95% confidence intervals.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example 1

### 1. Methods

### 1.1. Setting and Cohort Assembly

We conducted a multicenter observational retrospective cohort study at 36 AP-HP hospitals from the beginning of the epidemic in France, i.e. January 24^{th}, until May 1^{st}. We included all adults aged 18 years or over with a mental disorder who have been hospitalized in these medical centers for severe COVID-19. Mental disorder was defined as having at least one current International Statistical Classification of Diseases and Related Health Problems (ICD-10) diagnosis of mental disorder (F01-F99) during the visit or an ongoing prescription of any antidepressant, antipsychotic, or mood stabilizer (i.e. lithium or antiepileptic medications with mood stabilizing effects) for a psychiatric disorder at hospital admission. COVID-19 was ascertained by a positive reverse-transcriptase-polymerase-chain-reaction (RT-PCR) test from analysis of nasopharyngeal or oropharyngeal swab specimens. Severe COVID-19 was defined as having at least one of the following criteria at baseline [19-21]: respiratory rate >24 breaths/min or <12 breaths/min, resting peripheral capillary oxygen saturation in ambient air <90%, temperature >40° C., systolic blood pressure <100 mm Hg, lactate levels >2 mmol/L, or admission to an intensive care unit (ICU) within the first 24 hours form hospital admission.

AP-HP clinical Data Warehouse initiatives ensure patient information and informed consent regarding the different approved studies through a transparency portal in accordance with European Regulation on data protection and authorization n°1980120 from National Commission for Information Technology and Civil Liberties (CNIL). All procedures related to this work adhered to the ethical standards of the relevant national and institutional committees on human experimentation and with the Helsinki Declaration of 1975, as revised in 2008.

### 1.2. Data Sources

We used data from the AP-HP Health Data Warehouse (‘Entrepôt de Données de Santé (EDS)’). This warehouse contains all available clinical data on all inpatient visits for COVID-19 to 36 Greater Paris University hospitals. The data included patient demographic characteristics, vital signs, laboratory test and RT-PCR test results, medication administration data, medication lists during current and past hospitalizations in AP-HP hospitals, current diagnoses, discharge disposition, and death certificates.

### 1.3. Variables Assessed

We obtained the following data for each patient at the time of the hospitalization: sex; age; hospital; obesity; current smoking status; any medication prescribed according to compassionate use or as part of a clinical trial; current psychiatric disorder (i.e. ICD-10 diagnosis of substance use disorder, psychotic disorder, mood or anxiety disorder, delirium or dementia, and other psychiatric disorders); and any prescription for antidepressant, mood stabilizer, benzodiazepine or Z-drug, or antipsychotic medication. These variables are detailed in eMethods.

### 1.4. Psychotropic Medications Functionally Inhibiting Acid Sphingomyelinase (ASM)

FIASMA psychotropic medications were defined as psychotropic medications showing a substantial in vitro functional inhibition effect on ASM (i.e., a residual ASM activity lower than 50%), as described in detail elsewhere [4, 5, 12, 13]. FIASMA psychotropic medication use was defined as receiving at least one psychotropic FIASMA medication at study baseline, i.e. within the first 24 hours of hospital admission, and before the end of the index hospitalization, intubation or death. To minimize potential confounding effects of late prescription of FIASMA psychotropic medications, patients who received a FIASMA psychotropic medication 24 hours after hospital admission were excluded from the analyses. Finally, patients who received at study baseline any antipsychotic in ICU, possibly as an aid to oral intubation, were also excluded.

### 1.5. Primary Endpoint

Study baseline was defined as the date of hospital admission for COVID-19. The primary endpoint was the time from study baseline to intubation or death. For patients who died after intubation, the timing of the primary endpoint was defined as the time of intubation. Patients without an end-point event had their data censored on May 1^{st}, 2020.

### 1.6. Statistical Analysis

We calculated frequencies of baseline characteristics described above in patients receiving or not receiving a FIASMA psychotropic medication and compared them using standardized mean differences (SMD).

To examine the association between FIASMA psychotropic medication use and the endpoint of intubation or death, we performed Cox proportional-hazards regression models [22]. To help account for the nonrandomized prescription of psychotropic medications and reduce the effects of confounding, the primary analysis used propensity score analysis with inverse probability weighting (IPW) [23, 24]. Given the expected relatively limited sample size and the number of potentially influencing variables, a backward stepwise Cox regression was used to assess the importance of the covariates on the outcome, based on clinical meaningfulness and the Akaike Information Criterion (AIC) for model comparison [25]. Next, the individual propensities for receiving a FIASMA psychotropic medication were estimated using a multivariable logistic regression model including the variables from the model with the lowest AIC value. In the inverse-probability-weighted analyses, the predicted probabilities from the propensity-score models were used to calculate the stabilized inverse-probability-weighting weights [23]. The association between FIASMA psychotropic medication use and the endpoint was then estimated using an IPW Cox regression model. In case of unbalanced covariates, an IPW multivariable Cox regression model adjusting for the unbalanced covariates was also performed. Kaplan-Meier curves were performed using the inverse-probability-weighting weights [26, 27] and their pointwise 95% confidence intervals were estimated using the nonparametric bootstrap method [27].

We conducted two sensitivity analyses. First, we performed a multivariable Cox regression model including as covariates the same variables used in the IPW analysis. Second, we used a univariate Cox regression model in a matched analytic sample using a 1:1 ratio, based on the same variables used for the IPW analysis and the multivariable Cox regression analysis. To reduce the effects of confounding, optimal matching was used in order to obtain the smallest average absolute distance across all clinical characteristics between exposed patients and non-exposed matched controls [28].

We performed four additional exploratory analyses. First, we examined the relationships between each FIASMA psychotropic class (i.e. FIASMA antidepressants and antipsychotics) and each individual FIASMA molecule with the endpoint. Second, we examined within each psychotropic class (i.e. antidepressants and antipsychotics) the relationships of FIASMA and non-FIASMA molecules with the endpoint. Third, because of discrepancies in the potential FIASMA in vitro effect of venlafaxine, mirtazapine, and citalopram [4][5], we reproduced the main analyses while considering these molecules as FIASMAs. Finally, we reproduced the main analyses among all patients with mental disorder with and without clinical severity criteria at baseline.

For all associations, we performed residual analyses to assess the fit of the data, check assumptions, including proportional hazards assumption using proportional hazards tests and diagnostics based on weighted residuals [22, 29], and examined the potential influence of outliers. Because our main analysis focused on the association between FIASMA psychotropic medication use and the composite outcome of intubation or death among patients with mental disorder hospitalized for severe COVID-19, statistical significance was fixed a priori at two-sided p-value <0.05. Only if a significant protective association were found, we planned to perform additional exploratory analyses as described above. All analyses were conducted in R software version 2.4.3 (R Project for Statistical Computing).

### 2. Results

### 2.1. Characteristics of the Cohort

Of the 17,131 patients with a positive COVID-19 RT-PCR test who had been hospitalized for COVID-19, 1,963 (11.5%) were excluded because of missing data or their young age (i.e. less than 18 years old of age). Of 15,168 adult inpatients, 1,998 (13.2%) had a mental disorder diagnosis or an ongoing prescription of any antidepressant, antipsychotic, or mood stabilizer at hospital admission. Of these 1,998 patients, 827 (41.4%) had criteria for severe COVID-19. Of these 827 patients, 281 (34.0%) were excluded because they received a FIASMA psychotropic medication after 24 hours from hospital admission (N=277) or because they received an antipsychotic at baseline while being hospitalized in an ICU, possibly as an aid for intubation (N=5). Of the remaining 545 adult inpatients with mental disorder and severe COVID-19, 164 (30.1%) received a FIASMA psychotropic medication at baseline and 381 (69.9%) did not (FIG. 1).

PT-PCR test results were obtained after a median delay of 0.9 days (SD=9.4) from hospital admission date. This median delay was 0.9 days in the exposed group (SD=11.4) and the non-exposed group (SD=9.3).

Over a mean follow-up of 9.2 days (SD=12.5; median=6 days), 272 patients (50.0%) had an end-point event at the time of data cutoff on May 1^{st}. Among patients who received a FIASMA psychotropic medication, the mean follow-up was 12.0 days (SD=12.9, median=8 days), while it was of 8.9 days (SD=12.4, median=5 days) in those who did not.

Sex, hospital, number of medical conditions, delirium or dementia, any other mental disorder, and the prescription of any antidepressant, any antipsychotic, and any mood stabilizer were significantly associated with the endpoint of intubation or death. A backward stepwise Cox regression showed that a model including age, sex, hospital, obesity, and the number of medical conditions, was meaningful and associated with the lowest AIC value.

The distributions of patient characteristics included in the propensity and regression analyses according to FIASMA psychotropic medication use are shown in Table 1. In the full sample, FIASMA psychotropic medication use substantially differed according to age, sex, hospital, and number of medical conditions. After applying the propensity score weights, there were no differences (i.e. all SMD<0.1) in any characteristic. In the matched analytic sample using a 1:1 ratio, sex, and the number of medical conditions differed between groups (Table 1).

TABLE 1

Characteristics of patients with mental disorder and severe COVID-19 receiving or not receiving FIASMA psychotropic medications at baseline (N=545)

| | Exposed to any FIASMA (N=164) | Not exposed to any FIASMA (N=381) | Non-exposed matched group (N=164) | Exposed to any FIASMA vs. Not exposed | Exposed to any FIASMA vs. Not exposed | Exposed to any FIASMA vs. Non-exposed matched group |
|---|---|---|---|---|---|---|
| | | | | Crude analysis | Analysis weighted by inverse-probability-weighting weights | Matched analytic sample analysis |
| | N (%) | N (%) | N (%) | SMD | SMD | SMD |
| Age | | | | 0.197 | 0.037 | 0.022 |
| 18 to 50 years | 19 (11.6%) | 26 (6.82%) | 18 (48.6%) | | | |
| 51 to 70 years | 43 (26.2%) | 105 (27.6%) | 43 (50.0%) | | | |
| 71 to 80 years | 37 (22.6%) | 75 (19.7%) | 38 (50.7%) | | | |
| More than 80 years | 65 (39.6%) | 175 (45.9%) | 65 (50.0%) | | | |

| | | | | | | |
|---|---|---|---|---|---|---|
| Sex | | | | 0.260 | 0.008 | 0.159 |
| Women | 90 (54.9%) | 160 (42.0%) | 77 (46.1%) | | | |
| Men | 74 (45.1%) | 221 (58.0%) | 87 (54.0%) | | | |

| Hospital | | | | 0.267 | 0.024 | 0.068 |
|---|---|---|---|---|---|---|
| AP-HP Centre - Paris University, | | | | | | |
| Henri Mondor University Hospitals and at home hospitalization | 36 (22.0%) | 115 (30.2%) | 40 (52.6%) | | | |
| Universitaires Paris Seine-Saint-Denis | 45 (27.4%) | 77 (20.2%) | 46 (50.5%) | | | |
| AP-HP Paris Saclay University | 41 (25.0%) | 113 (29.7%) | 38 (48.1%) | | | |
| AP-HP Sorbonne University | 42 (25.6%) | 76 (19.9%) | 40 (48.8%) | | | |
| Obesity^{a} | | | | 0.129 | 0.001 | 0.162 |
| Yes | 42 (25.6%) | 77 (20.2%) | 31 (42.5%) | | | |
| No | 122 (74.4%) | 304 (79.8%) | 133 (52.2%) | | | |
| Number of medical conditions^{b} | | | | 0.321 | 0.015 | 0.029 |
| 0 | 42 (25.6%) | 51 (13.4%) | 40 (48.8%) | | | |
| 1 | 17 (10.4%) | 37 (9.71%) | 17 (50.0%) | | | |
| 2 or more | 105 (64.0%) | 293 (76.9%) | 107 (50.5%) | | | |

^{a} Defined as having a body-mass index higher than 30 kg/m² or an International Statistical Classification of Diseases and Related Health Problems (ICD-10) diagnosis code for obesity (E66.0, E66.1, E66.2, E66.8, E66.9).
^{b} Assessed using ICD-10 diagnosis codes for diabetes mellitus (E11), diseases of the circulatory system (I00-I99), diseases of the respiratory system (J00-J99), neoplasms (C00-D49), diseases of the blood and blood-forming organs and certain disorders involving the immune mechanism (D5-D8), frontotemporal dementia (G31.0), peptic ulcer (K27), diseases of liver (K70-K95), hemiplegia or paraplegia (G81-G82), acute kidney failure or chronic kidney disease (N17-N19), and HIV (B20).
SMD>0.1 are in bold and indicate substantial difference.
Abbreviation: SMD, standardized mean difference.

### 2.2. Study Endpoint

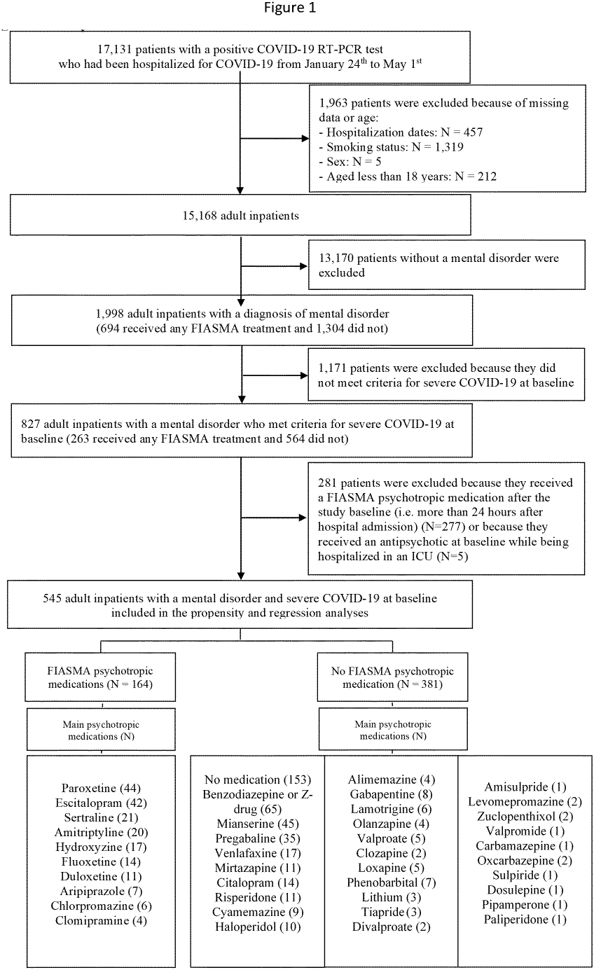

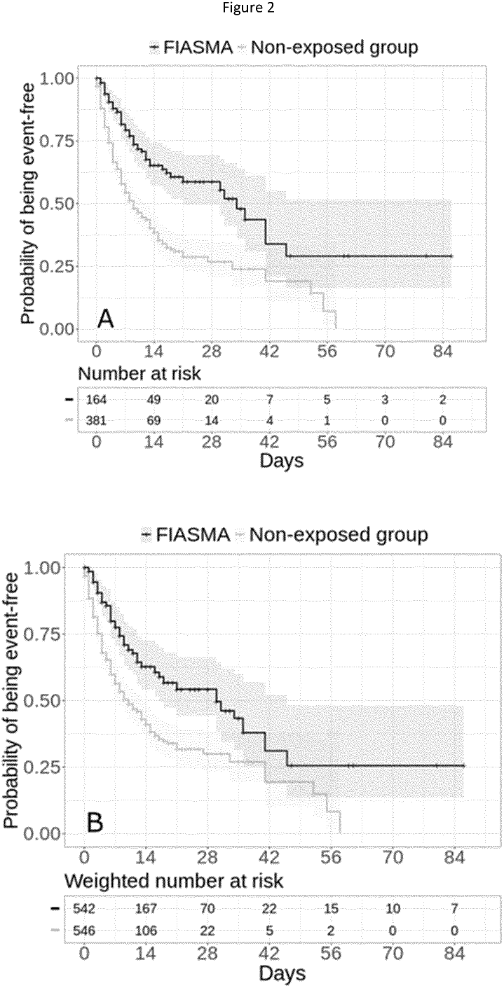

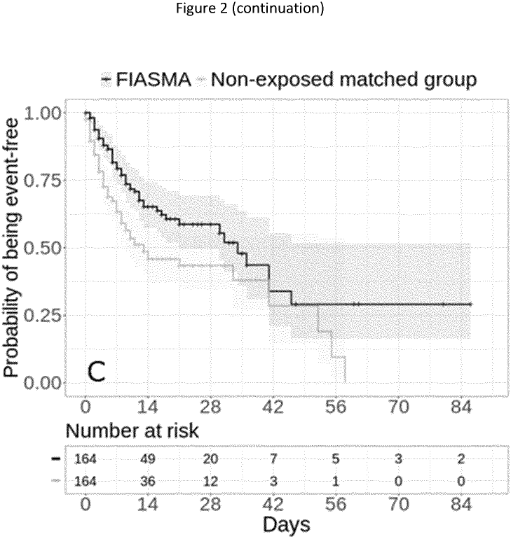

The endpoint of death occurred in 57 patients (34.8%) who received a FIASMA psychotropic medication at baseline and 215 patients (56.4%) who did not. The crude, unadjusted analysis (hazard ratio (HR)=0.42; 95% CI=0.31-0.57; p<0.001) and the primary analysis with inverse probability weighting (HR=0.50; 95% CI=0.37-0.67; p<0.001) showed a significant association between FIASMA psychotropic medication use and reduced risk of intubation or death (FIG. 2; Table 2). A post-hoc analysis indicated that we had 80% power in the crude analysis to detect a hazard ratio of at least 0.60 / 1.71.

In sensitivity analyses, the multivariable Cox regression model also yielded a significant association (HR=0.49; 95% CI=0.36-0.67; p<0.001), as did the Cox regression model in a matched analytic sample using a 1:1 ratio adjusted for unbalanced covariates, i.e. sex, and number of medical conditions (HR=0.55; 95% CI=0.39-0.77; p=0.001) (Table 2).

TABLE 2

Association between FIASMA psychotropic medication use at baseline and risk of intubation or death among patients with mental disorder hospitalized for severe COVID-19

| | Number of events / Number of patients | Crude Cox regression analysis | Multivariable Cox regression analysis ^{a} | Analysis weighted by inverse-probability-weighting weights ^{a} | Number of events / Number of patients in the matchedgroups | Univariate Cox regression in a 1:1 ratio matched analytic sample ^{a} | Cox regression in a 1:1 ratio matched analytic sample ^{a} adjusted for unbalanced covariates ^{b} |
|---|---|---|---|---|---|---|---|
| | N (%) | HR (95% CI; p-value) | HR (95% CI; p-value) | HR (95% CI; p-value) | N (%) | HR (95% CI; p-value) | HR (95% CI; p-value) |
| No FIASMA psychotropic medication | 215/381 (56.4%) | Ref. | Ref. | Ref. | 77 / 164 (47%) | Ref. | Ref. |
| Any FIASMA psychotropic medication | 57/164 (34.8%) | 0.42 (0.31 - 0.57; <0.001*) | 0.49 (0.36 - 0.67; <0.001*) | 0.50 (0.37 - 0.67; <0.001*) | 57 / 164 (34.8%) | 0.65 (0.45 - 0.93; 0.019*) | 0.55 (0.39 - 0.77; 0.001*) |

^{a} Adjusted for age, sex, hospital, obesity, and number of medical conditions.
^{b} Adjusted for sex and obesity.
*Two-sided p-value is significant (p<0.05).
Abbreviations: HR, hazard ratio, CI confidence interval.

Additional exploratory analyses showed that FIASMA antidepressant use was significantly associated with reduced risk of intubation or death across all analyses (not shown). FIASMA antipsychotic use was significantly associated with reduced risk only in the multivariable Cox regression model and in the Cox regression model in a matched analytic sample using a 1:2 ratio adjusted for unbalanced covariates, possibly because of limited statistical power due to the limited number of patients receiving a FIASMA antipsychotic at hospital admission (N=13) (not shown). Hazard ratios were lower than 1 for most individual FIASMA molecules, but none of them reached statistical significance across all main and sensitivity analyses, except for hydroxyzine and escitalopram, possibly because of limited statistical power due to individual sample sizes ≤42 patients. Patients receiving a FIASMA antidepressant at baseline (N=148) had a significantly reduced risk of intubation or death compared with those receiving a non-FIASMA antidepressant at baseline (N=83) (not shown). Adjusted analyses between FIASMA and non-FIASMA antipsychotics and antihistaminic medications could not be performed due to the insufficient number of events (i.e., <5) in the FIASMA groups (not shown). Finally, reproducing the main analyses among all patients with mental disorder with and without clinical severity criteria at baseline did not alter the significance of our results (not shown), as for the main analyses considering venlafaxine, mirtazapine and citalopram as FIASMA antidepressants (not shown).

### 3. Discussion

We found that FIASMA psychotropic medication use was significantly and substantially associated with reduced risk of intubation or death among patients with mental disorder hospitalized for severe COVID-19, and that this association was not specific to one FIASMA psychotropic class or medication in this population. These findings are in line with prior preclinical [4, 6] and clinical [7-9] evidence that FIASMA antidepressant medications may substantially prevent cells from being infected with SARS-CoV-2 in vitro [4, 6], and that FIASMA antidepressant medications and the FIASMA hydroxyzine at their usual respective antidepressant and antihistaminic doses, may reduce mortality among patients hospitalized for COVID-19 [7-9].

However, several alternative mechanisms could be proposed to explain this association. First, antiviral effects, i.e. inhibition of viral replication, of FIASMA medications might underlie this relationship, as suggested by a recent in-vitro study [4] for fluoxetine. However, inhibition of viral replication was not observed for other selective serotonin reuptake inhibitors (SSRIs) and FIASMA medications, including paroxetine and escitalopram. Second, many antidepressants (except for example sertraline and paroxetine) have high affinity for Sigma-1 receptors (S1R) [30, 31], and SSRIs have been suggested to have potential value in regulating inflammation by inhibiting cytokine production in COVID-19 [7, 32]. Because most FIASMA psychotropic antidepressants are S1R agonists, this mechanism might have overlapped their inhibition effect on ASM. However, when examining the association between several FIASMA psychotropic medications with low or no affinity for S1R (i.e., sertraline, paroxetine, duloxetine, aripiprazole and chlorpromazine) [30, 33-35] and the endpoint, main results remained statistically significant, suggesting that inhibition of ASM could underlie this association independently of S1R. Finally, this association may be partly mediated by the anti-inflammatory effects of FIASMA psychotropic medications, which could be explained by inhibition of the ASM in endothelial cells and the immune system, and might be independent of Sigma-1 receptors. First, a recent meta-analysis [36] of studies conducted in individuals with major depressive disorder following antidepressant treatment, mostly including SSRIs, supports that, overall, antidepressants may be associated with decreased plasma levels of 4 of 16 tested inflammatory mediators, including IL-10, TNF-α, and CCL-2, which are associated with COVID-19 severity [37], as well as IL-6, which is highly correlated with disease mortality [37, 38]. Second, prior in vitro and in vivo studies [39-41] suggest that antipsychotics may induce anti-inflammatory effect dependently on glia activation, and that this activity may not be shared by all antipsychotics. However, this anti-inflammatory effect was observed for both FIASMA antipsychotics (e.g. chlorpromazine) and non-FIASMA ones (e.g., haloperidol and risperidone). If the association between FIASMA psychotropic medication use and reduced risk of intubation or death were confirmed, future studies aiming at disentangling these potentially interrelated mechanisms are needed.

In this multicenter observational retrospective study, FIASMA psychotropic medication use at baseline was significantly associated with a 50% reduction in risk of intubation or death among adult patients with mental disorder hospitalized for severe COVID-19. These findings suggest the usefulness of the ASM/ceramide system framework in COVID-19.

### Example 2

### 1. Methods

### 1.1. Setting and Cohort Assembly

We conducted a multicenter observational retrospective cohort study at 36 AP-HP hospitals from the beginning of the epidemic in France, i.e. January 24^{th}, until May 1^{st} 2020. We included all adults aged 18 years or over who have been hospitalized in these medical centers for severe COVID-19. COVID-19 was ascertained by a positive reverse-transcriptase-polymerase-chain-reaction (RT-PCR) test from analysis of nasopharyngeal or oropharyngeal swab specimens. Severe COVID-19 at admission was defined as having at least one of the following criteria: respiratory rate > 24 breaths/min or < 12 breaths/min, resting peripheral capillary oxygen saturation in ambient air < 90%, temperature > 40° C., systolic blood pressure < 100 mm Hg or high lactate levels [19-21]. AP-HP clinical Data Warehouse initiatives ensure patient information and informed consent regarding the different approved studies through a transparency portal in accordance with European Regulation on data protection and authorization n°1980120 from National Commission for Information Technology and Civil Liberties (CNIL).

### 1.2. Data Sources

We used data from the AP-HP Health Data Warehouse (‘Entrepôt de Données de Santé (EDS)’). This warehouse contains all available clinical data on all inpatient visits for COVID-19 to any Greater Paris University hospital. The data included patient demographic characteristics, vital signs, laboratory test and RT-PCR test results, medication administration data, medication lists during current and past hospitalizations in AP-HP hospitals, current diagnoses, discharge disposition, and death certificates.

### 1.3. Variables Assessed

We obtained the following data for each patient at the time of the hospitalization: sex; age, which was categorized into 4 classes based on the OpenSAFELY study results [45] (i.e. 18-50, 51-70, 71-80, 81+); hospital, which was categorized into 4 classes following the administrative clustering of AP-HP hospitals in Paris and its suburbs based on their geographical location (i.e., AP-HP Centre - Paris University, Henri Mondor University Hospitals and at home hospitalization; AP-HP Nord and Hôpitaux Universitaires Paris Seine-Saint-Denis; AP-HP Paris Saclay University; and AP-HP Sorbonne University); obesity, which was defined as having a body mass index higher than 30 kg/m² or an International Statistical Classification of Diseases and Related Health Problems (ICD-10) diagnosis code for obesity (E66.0, E66.1, E66.2, E66.8, E66.9); self-reported current smoking status; and any medication prescribed according to compassionate use or as part of a clinical trial (e.g. hydroxychloroquine, azithromycin, remdesivir, tocilizumab, sarilumab, or dexamethasone). To take into account possible confounding by indication bias for FIASMA medication, we recorded whether patients had any current diagnosis, based on ICD-10 diagnosis codes, for neoplasms and diseases of the blood (C00-D89); mental disorders (F01-F99); diseases of the nervous system (G00-G99); cardiovascular disorders (I00-I99); respiratory disorders (J00-J99); digestive disorders (K00-K95); dermatological disorders (L00-L99); diseases of the musculoskeletal system (M00-M99); diseases of the genitourinary system (N00-N99); endocrine disorders (E00-E89); and eye-ear-nose-throat disorders (H00-H95).

All medical notes and prescriptions are computerized in Greater Paris University hospitals. Medications including their dosage, frequency, date, and mode of administration were identified from medication administration data or scanned handwritten medical prescriptions, through two deep learning models based on BERT contextual embeddings [46], one for the medications and another for their mode of administration. The model was trained on the APmed corpus [47], a previously annotated dataset for this task. Extracted medications names were then normalized to the Anatomical Therapeutic Chemical (ATC) terminology using approximate string matching.

### 1.4. Medications With Functional Inhibition Effect on Acid Sphingomyelinase (ASM)

FIASMA medications were defined as having a substantial in vitro functional inhibition effect on ASM (i.e., a residual ASM activity lower than 50%), as detailed elsewhere [4, 5, 12, 13], and included: FIASMA alimentary tract and metabolism medication (i.e. loperamide); FIASMA cardiovascular system medication, subdivided into calcium channel blockers (i.e. amlodipine) and other cardiovascular medications (i.e. amiodarone and carvedilol); FIASMA nervous system medication, subdivided into psychoanaleptic medications (i.e. amitriptyline, clomipramine, duloxetine, escitalopram, fluoxetine, paroxetine, sertraline and hydroxyzine) and psycholeptic medications (i.e. aripiprazole and chlorpromazine); and FIASMA respiratory system medication (i.e. desloratadine), according to their ATC index code [48]. FIASMA medication use was defined as receiving at least one FIASMA medication at study baseline, i.e. within the first 24 hours of hospital admission, and before the end of the index hospitalization, intubation or death. To minimize potential confounding effects of late prescription of FIASMA medications, patients who received a FIASMA medication 24 hours after hospital admission were excluded from the analyses. Patients who received at baseline an antipsychotic or a benzodiazepine in ICU, possibly as an aid to oral intubation, were also excluded.

### 1.5. Primary Endpoint

Study baseline was defined as the date of hospital admission for COVID-19. The primary endpoint was the time from study baseline to intubation or death. For patients who died after intubation, the timing of the primary endpoint was defined as the time of intubation. Patients without an end-point event had their data censored on May 1^{st}, 2020.

### 1.6. Statistical Analysis

We calculated frequencies of all baseline characteristics described above in patients receiving or not receiving a FIASMA medication and compared them using standardized mean differences (SMD).

To examine the association between FIASMA medication use and the endpoint of intubation or death, we performed Cox proportional-hazards regression models [22]. To help account for the nonrandomized prescription of medications and reduce the effects of confounding, the primary analysis used propensity score analysis with inverse probability weighting (IPW) [23, 24]. The individual propensities for receiving a FIASMA medication were estimated using a multivariable logistic regression model that included patient characteristics (i.e., sex, age, hospital, obesity, current smoking status, and significant medical conditions). In the inverse-probability-weighted analyses, the predicted probabilities from the propensity-score models were used to calculate the stabilized inverse-probability-weighting weights [23]. The association between FIASMA use and the endpoint was then estimated using an IPW Cox regression model. In case of non-balanced covariates, an IPW multivariable Cox regression model adjusting for the non-balanced covariates was also performed. Kaplan-Meier curves were performed using the inverse-probability-weighting weights [26,27] and their pointwise 95% confidence intervals were estimated using the nonparametric bootstrap method [27, 49].

We conducted two sensitivity analyses. First, we performed a multivariable Cox regression model including as covariates the same variables used in the IPW analysis. Second, we used a univariate Cox regression model in a matched analytic sample using a 1:1 ratio, based on the same variables used for the IPW analysis and the multivariable Cox regression analysis. To reduce the effects of confounding, optimal matching was used in order to obtain the smallest average absolute distance across all clinical characteristics between exposed patients and non-exposed matched controls [28].

We performed three additional exploratory analyses. First, we examined the relationships between each FIASMA class (i.e. alimentary tract and metabolism, cardiovascular system, cardiovascular system calcium channel blockers, other cardiac therapy, nervous system, nervous system psychoanaleptic, nervous system psycholeptic and respiratory system) and each individual FIASMA molecule with the endpoint. Second, we performed additional Cox proportional-hazard regression analyses to compare the potential effect of each FIASMA class to that of an active comparator, i.e. paracetamol. Finally, we reproduced the main analyses among all patients with and without clinical severity criteria at baseline.

For all associations, we performed residual analyses to assess the fit of the data, check assumptions, including proportional hazards assumption using proportional hazards tests and diagnostics based on weighted residuals [22, 29], and examined the potential influence of outliers. To improve the quality of result reporting, we followed the recommendations of The Strengthening the Reporting of Observational Studies in Epidemiology (STROBE) Initiative [49]. Statistical significance was fixed a priori at two-sided p-value <0.05. All analyses were conducted in R software version 2.4.3 (R Project for Statistical Computing).

### 2. Results

### 2.1. Characteristics of the Cohort

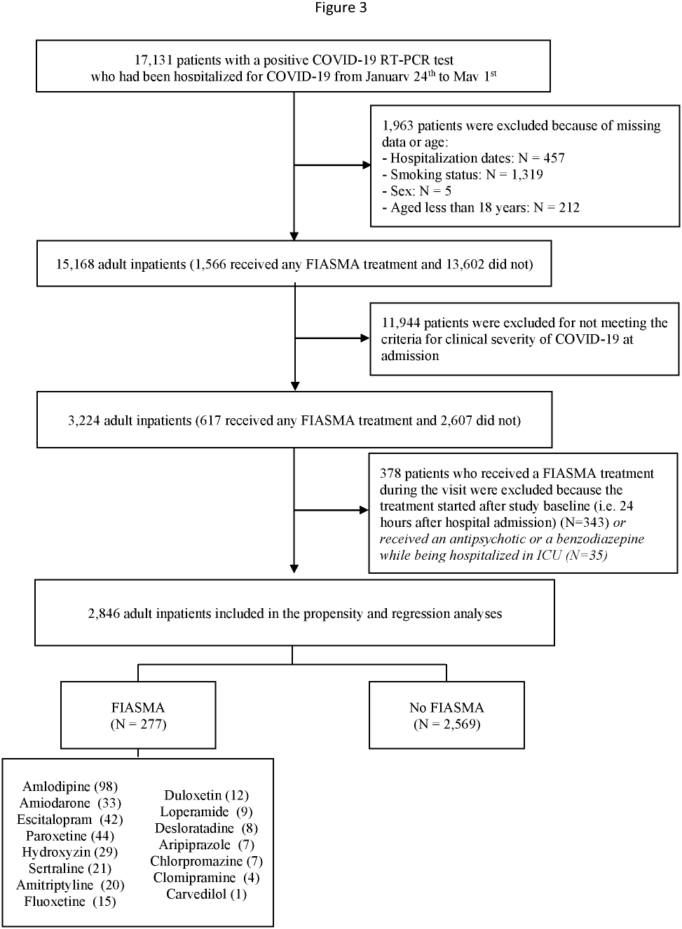

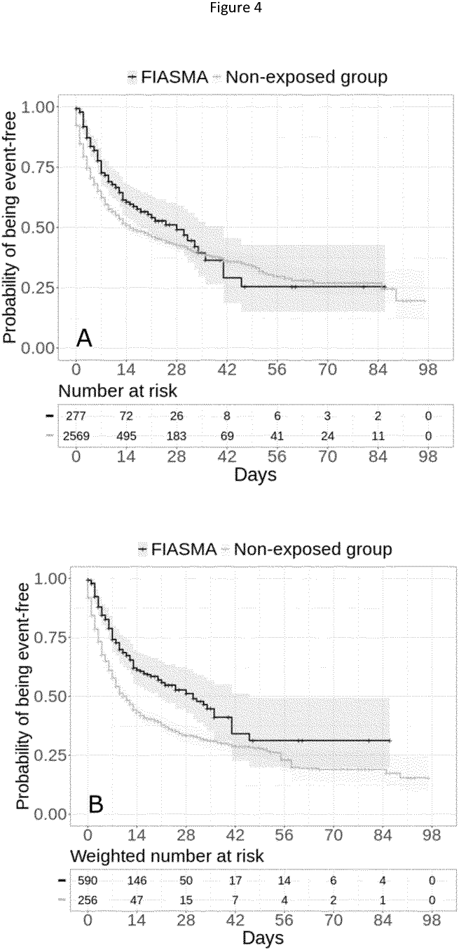

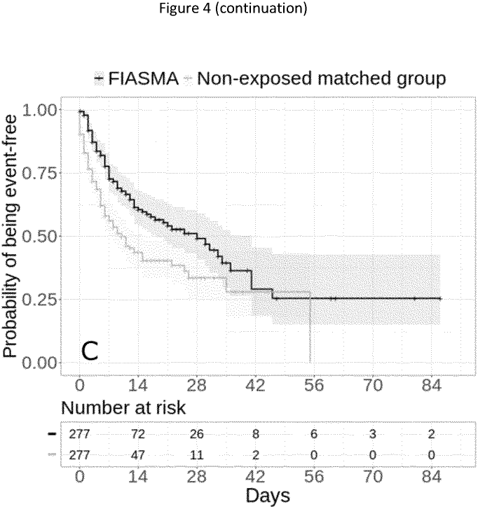

Of the 17,131 patients with a positive COVID-19 RT-PCR test who had been hospitalized for COVID-19, 1,963 patients (11.5%) were excluded because of missing data or their young age (i.e. less than 18 years old of age). Of these 15,168 patients, 3,224 (21.3%) had criteria for severe COVID-19. Of these 3,224 patients, 378 (11.7%) were excluded because they received a FIASMA medication after 24 hours from hospital admission (N=343) or because they received an antipsychotic or a benzodiazepine at baseline while being hospitalized in an ICU, possibly as an aid for intubation (N=35). Of the remaining 2,846 adult patients, 277 (9.7%) received a FIASMA medication in the first 24 hours of hospitalization (FIG. 3).

PT-PCR test results were obtained after a median delay of 0.9 days (SD=9.4) from hospital admission date. This median delay was of 0.9 days in the exposed group (SD=11.4) and in the non-exposed group (SD=9.3).

Over a mean follow-up of 9.2 days (SD=12.5; median=6 days), 1,168 patients (41.0%) had an end-point event at the time of data cutoff on May 1st. Among patients who received a FIASMA, the mean follow-up was 12.0 days (SD=12.9, median=8 days), while it was of 8.9 days (SD=12.4, median=5 days) in those who did not.

All patient characteristics except for current smoking status, diseases of the musculoskeletal system, and Eye-Ear-Nose-Throat disorders were independently and significantly associated with the endpoint. A multivariable Cox regression model showed that sex, hospital, obesity, medication according to compassionate use or as part of a medical trial, other infectious diseases, neoplasms and diseases of the blood, cardiovascular disorders, respiratory disorders, and diseases of the genitourinary system were significantly and independently associated with the endpoint (Table 3).

TABLE 3

Characteristics of patients with severe COVID-19 receiving or not receiving FIASMA medications at baseline (N=2846).

| | Exposed to any FIASMA (N=277) | Not exposed to any FIASMA (N=2569) | Non-exposed matched group (N=277) | Exposed to any FIASMA vs. Not exposed | Exposed to any FIASMA vs. Not exposed | Exposed to any FIASMA vs. Non-exposed matched group |
|---|---|---|---|---|---|---|
| | | | | Crude analysis | Analysis weighted by inverse-probability-weighting weights | Matched analytic sample analysis |
| | N (%) | N (%) | N (%) | SMD | SMD | SMD |
| Age | | | | 0.369 | 0.097 | 0.095 |
| 18 to 50 years | 29 (10.5%) | 493 (19.2%) | 29 (10.5%) | | | |
| 51 to 70 years | 88 (31.8%) | 1027 (40.0%) | 99 (35.7%) | | | |
| 71 to 80 years | 63 (22.7%) | 457 (17.8%) | 55 (19.9%) | | | |
| More than 80 years | 97 (35.0%) | 592 (23.0%) | 94 (33.9%) | | | |
| Sex | | | | 0.224 | 0.034 | 0.094 |
| Women | 131 (47.3%) | 933 (36.3%) | 118 (42.6%) | | | |
| Men | 146 (52.7%) | 1636 (63.7%) | 159 (57.4%) | | | |
| AP-HP Centre - Paris University, Henri Mondor University Hospitals and at home hospitalization | 62 (22.4%) | 660 (25.7%) | 70 (25.3%) | | | |
| AP-HP Nord and Hôpitaux Universitaires Paris Seine-Saint-Denis | 76 (27.4%) | 813 (31.6%) | 68 (24.5%) | | | |
| AP-HP Paris Saclay University | 63 (22.7%) | 561 (21.8%) | 68 (24.5%) | | | |
| AP-HP Sorbonne University | 76 (27.4%) | 535 (20.8%) | 71 (25.6%) | | | |
| Yes | 67 (24.2%) | 515 (20.0%) | 63 (22.7%) | | | |
| No | 210 (75.8%) | 2054 (80.0%) | 214 (77.3%) | | | |
| Yes | 46 (16.6%) | 310 (12.1%) | 40 (14.4%) | | | |
| No | 231 (83.4%) | 2259 (87.9%) | 237 (85.6%) | | | |
| Medication according to compassionate use or as part of a clinical trial ^{c} | | | | 0.040 | 0.020 | 0.024 |
| Yes | 83 (30.0%) | 723 (28.1%) | 80 (28.9%) | | | |
| No | 194 (70.0%) | 1846 (71.9%) | 197 (71.1%) | | | |
| Yes | 55 (19.9%) | 301 (11.7%) | 50 (18.1%) | | | |
| No | 222 (80.1%) | 2268 (88.3%) | 227 (81.9%) | | | |
| Yes | 46 (16.6%) | 293 (11.4%) | 41 (14.8%) | | | |
| No | 231 (83.4%) | 2276 (88.6%) | 236 (85.2%) | | | |
| Yes | 70 (25.3%) | 270 (10.5%) | 60 (21.7%) | | | |
| No | 207 (74.7%) | 2299 (89.5%) | 217 (78.3%) | | | |
| Yes | 49 (17.7%) | 243 (9.5%) | 41 (14.8%) | | | |
| No | 228 (82.3%) | 2326 (90.5%) | 236 (85.2%) | | | |
| Yes | 147 (53.1%) | 873 (34.0%) | 144 (52.0%) | | | |
| No | 130 (46.9%) | 1696 (66.0%) | 133 (48.0%) | | | |
| Yes | 195 (70.4%) | 1509 (58.7%) | 203 (73.3%) | | | |
| No | 82 (29.6%) | 1060 (41.3%) | 74 (26.7%) | | | |
| Yes | 42 (15.2%) | 192 (7.5%) | 35 (12.6%) | | | |
| No | 235 (84.8%) | 2377 (92.5%) | 242 (87.4%) | | | |
| Yes | 14 (5.1%) | 57 (2.2%) | 14 (5.1%) | | | |
| No | 263 (94.9%) | 2512 (97.8%) | 263 (94.9%) | | | |
| Yes | 22 (7.9%) | 121 (4.7%) | 22 (7.94%) | | | |
| No | 255 (92.1%) | 2448 (95.3%) | 255 (92.1%) | | | |
| Yes | 76 (27.4%) | 353 (13.7%) | 70 (25.3%) | | | |
| No | 201 (72.6%) | 2216 (86.3%) | 207 (74.7%) | | | |
| Yes | 134 (48.4%) | 908 (35.3%) | 135 (48.7%) | | | |
| No | 143 (51.6%) | 1661 (64.7%) | 142 (51.3%) | | | |
| Yes | 12 (4.3%) | 47 (1.8%) | 12 (4.33%) | | | |
| No | 265 (95.7%) | 2522 (98.2%) | 265 (95.7%) | | | |

^{a} Defined as having a body-mass index higher than 30 kg/m² or an International Statistical Classification of Diseases and Related Health Problems (ICD-10) diagnosis code for obesity (E66.0, E66.1, E66.2, E66.8, E66.9).
^{b} Current Smoking status was self-reported.
^{c} Any medication prescribed as part of a clinical trial or according to compassionate use (e.g., hydroxychloroquine, azithromycin, remdesivir, tocilizumab, sarilumab or dexamethasone).
^{d} Assessed using ICD-10 diagnosis codes for certain infectious and parasitic diseases (A00-B99).
^{e} Assessed using ICD-10 diagnosis codes for neoplasms (C00-D49) and diseases of the blood and blood-forming organs and certain disorders involving the immune mechanism (D50-D89).
^{f} Assessed using ICD-10 diagnosis codes for mental, behavioural and neurodevelopmental disorders (F01-F99).
^{g} Assessed using ICD-10 diagnosis codes for diseases of the nervous system (G00-G99).
^{h} Assessed using ICD-10 diagnosis codes for diseases of the circulatory system (I00-I99).
ⁱ Assessed using ICD-10 diagnosis codes for diseases of the respiratory system (J00-J99).
^{j} Assessed using ICD-10 diagnosis codes for diseases of the digestive system (K00-K95).
^{k} Assessed using ICD-10 diagnosis codes for diseases of the skin and subcutaneous tissue (L00-L99).
^{l} Assessed using ICD-10 diagnosis codes for diseases of the musculoskeletal system and connective tissue (M00-M99).
^{m} Assessed using ICD-10 diagnosis codes for diseases of the genitourinary system (N00-N99).
ⁿ Assessed using ICD-10 diagnosis codes for endocrine, nutritional and metabolic diseases (E00-E89).
^{o} Assessed using ICD-10 diagnosis codes for diseases of the eye and adnexa (H00-H59) and diseases of the ear and mastoid process (H60-H95).
SMD>0.1 indicates substantial difference.
Abbreviation: SMD, standardized mean difference.

The distributions of patient characteristics according to FIASMA use are shown in Table 4. In the full sample, FIASMA use substantially differed according to all patient characteristics, except for medication according to compassionate use or as part of a medical trial, and the direction of the associations indicated older age and a higher medical severity of patients receiving any FIASMA. There were no substantial differences in any characteristic after applying the propensity score weights and in the matched analytic sample (Table 4).

TABLE 4

Association between FIASMA medication use at baseline and risk of intubation or death among patients hospitalized for severe COVID-19.

| | Number of events / Number of patients | Crude Cox regression analysis | Multivariable Cox regression analysis | Analysis weighted by inverse-probability-weighting weights | Number of events / Number of patients in the matched groups | Univariate Cox regression in a 1:1 ratio matched analytic sample |
|---|---|---|---|---|---|---|
| | N (%) | HR (95% CI; p-value) | HR (95% CI; p-value) | HR (95% CI; p-value) | N (%) | HR (95% CI; p-value) |
| No FIASMA | 1064 / 2569 (41.4%) | Ref | Ref | Ref | 137/277 (49.5%) | Ref |
| Any FIASMA | 104/277 (37.5%) | 0.71 (0.58 - 0.87; 0.001*) | 0.66 (0.53 - 0.83; <0.001*) | 0.58 (0.46 - 0.72; <0.001*) | 104/277 (37.5%) | 0.55 (0.43 - 0.73; <0.001*) |

*Two-sided p-value is significant (p<0.05).
Abbreviation: HR. hazard ratio; CI, confidence interval.

### 2.2. Study Endpoint

The endpoint of death occurred in 104 patients (37.5%) who received a FIASMA medication at baseline and 1,060 patients (41.4%) who did not. Both the crude unadjusted analysis (HR=0.71; 95%CI=0.58-0.87; <0.01) and the primary analysis with inverse probability weighting (HR=0.58; 95%CI=0.46-0.72; p<0.001) showed a significant association between FIASMA medication use and reduced risk of intubation or death (FIG. 2; Table 4). A post-hoc analysis indicated that we had 80% power in the crude analysis to detect a hazard ratio of at least 0.82 / 1.21.

In sensitivity analyses, the multivariable Cox regression model in the full sample also showed a significant association (HR=0.66; 95%CI=0.53-0.83; p<0.001), as did the univariate Cox regression model in a matched analytic sample using a 1:1 ratio (HR=0.55; 95%CI=0.43-0.73; p<0.001) (Table 3).

Additional exploratory analyses showed that FIASMA nervous system medication use, and specifically FIASMA psychoanaleptic medication use, was significantly associated with decreased risk of intubation or death across all analyses (not shown). FIASMA cardiovascular system medication use, and specifically FIASMA calcium channel blockers medication use, was significantly and negatively associated with the outcome in the primary IPW analysis, multivariable analysis, and IPW analysis adjusted for unbalanced covariates. Hazard ratios were lower than 1 for most individual FIASMA molecules, but none of them reached statistical significance across all main and sensitivity analyses, except for hydroxyzine and escitalopram (Table 5). Patients receiving at baseline a FIASMA calcium channel blocker medication, a FIASMA nervous system medication and a FIASMA nervous system psychoanaleptic medication had a significantly reduced risk of intubation or death compared with those receiving paracetamol at baseline (Table 6). Finally, reproducing the main analyses among all patients with and without clinical severity criteria at baseline did not alter the significance of our results (not shown).

TABLE 5

Association of individual FIASMA classes and molecules at baseline with the risk of intubation or death among patients hospitalized for severe COVID-19

| | Number of events / Number of patients | Crude Cox regression analysis | Multivariable Cox regression analysis ^{a} | Analysis weighted by inverse-probability-weighting weights ^{a} | Analysis weighted by inverse-probability-weighting weights ^{a} adjusted for unbalanced covariates | Number of events / Number of patients in the matched control groups | Univariate Cox regression in a 1:2 ratio matched analytic sample ^{a} | Cox regression in a 1:2 ratio matched analytic sample^{a} adjusted for unbalanced covariates |
|---|---|---|---|---|---|---|---|---|
| | N (%) | HR (95 CI; p-value) | HR (95 CI; p-value) | HR (95 CI; p-value) | HR (95 CI; p-value) | N (%) | HR (95 CI; p-value) | HR (95 CI; p-value) |
| No FIASMA medication | 1064 / 2569 (41.4) | Ref. | Ref. | Ref. | Ref. | Ref. | Ref. | Ref. |
| FIASMA Alimentary tract and metabolism | 2 / 9 (22.2) | 0.39 (0.10 -1.56; 0.182) | 0.25 (0.05 -1.33; 0.104) | 0.15 (0.02 -1.21; 0.075) | NA | 11 / 18 (61.1) | 0.24 (0.05 -1.12; 0.070) | NA |
| Loperamide | 2 / 9 (22.2) | 0.39 (0.10 -1.56; 0.182) | 0.25 (0.05 -1.33; 0.104) | 0.15 (0.02 -1.21; 0.075) | NA | 11 / 18 (61.1) | 0.24 (0.05 -1.12; 0.070) | NA |
| FIASMA cardiovascular system | 54/125 (43.2) | 1.07 (0.81 -1.41; 0.650) | 0.82 (0.64 -1.06; 0.135) | 0.61 (0.45 -0.81; <0.001*) | 0.61 (0.46 -0.83; 0.001*) ^{a} | 129 / 250 (51.6) | 0.80 (0.58 -1.10; 0.169) | 0.83 (0.62 -1.13; 0.238) |
| FIASMA cardiovascular ststem: calcium channel blockers | 38 / 97 (39.2) | 0.88 (0.61 -1.27; 0.510) | 0.70 (0.49 -0.98; 0.037*) | 0.56 (0.39 -0.79; <0.001*) | 0.68 (0.49 -0.94; 0.020*) ^{c} | 97 / 194 (50.0) | 0.74 (0.48 -1.16; 0.190) | 0.75 (0.51 -1.11; 0.149) ^{d} |
| Amlodipine | 38 / 97 (39.2) | 0.88 (0.61 -1.27; 0.510) | 0.70 (0.49 -0.98; 0.037*) | 0.56(0.39 -0.79; <0.001*) | 0.68 (0.49 -0.94; 0.020*) ^{e} | 97/194 (50.0) | 0.74 (0.48 -1.16; 0.190) | 0.75 (0.51 -1.11; 0.149) ^{f} |
| FIASMA cardiovascular system: other | 19 / 34 (55.9) | 1.66 (1.21 -2.28; 0.002*) | 1.27 (0.96 -1.69; 0.100) | NA | NA | 37 / 68 (54.4) | 0.91 0.52 -1.59; 0.748) | 0.80 (0.44 -1.46; 0.469) ^{g} |
| Amiodarone | 18 / 33 (54.5) | 1.61 (1.14 -2.27; 0.007*) | 1.26 (0.93 -1.70; 0.140) | NA | NA | 37 / 66 (56.1) | 0.90 (0.51 -1.58; 0.712) | 0.80 (0.44 -1.48; 0.479) ^{h} |
| Carvedilol | 1 / 1 (100) | NA | NA | NA | NA | NA | NA | NA |
| FIASMA nervous system | 61 / 175 (34.9) | 0.62 (0.48 -0.80; <0.001*) | 0.65 (0.49 -0.88; <0.001*) | 0.51 (0.38 -0.69; <0.001*) | 0.49 (0.37 -0.64; <0.001*) ⁱ | 173 / 350 (49.4) | 0.60 (0.44 -0.82; 0.002*) | 0.61 (0.45 -0.83; 0.002* |
| FIASMA nervous system: psychoanaleptic | 59 / 169 (34.9) | 0.62 (0.47 -0.80; <0.001*) | 0.65 (0.48 -0.87; <0.001*) | 0.51 (0.37 -0.70; <0.001*) | 0.48 (0.36 -0.63; <0.001*) k | 169 / 338 (50.0) | 0.58 (0.42 -0.80; <0.001*) | 0.60 (0.44 -0.82; 0.001* |
| Amitriptyline | 8 / 20 (40.0) | 0.65 (0.33 -1.31; 0.229) | 0.54 (0.25 -1.17; 0.120) | 0.61 (0.29 -1.28; 0.188) | NA | 21 / 40 (52.5) | 0.54 (0.24 -1.21; 0.134) | 0.61 (0.27 -1.36; 0.227) ^{m} |
| Clomipramine | 1 / 4 (25.0) | 0.58 (0.08 -4.14; 0.589) | NA | NA | NA | NA | NA | NA |
| Duloxetine | 5/12 (41.7) | 0.69 (0.29 -1.67; 0.413) | 0.65 (0.25 -1.64; 0.358) | 0.50 (0.20 -1.29; 0.151) | NA | 14 / 24 (58.3) | 0.44 (0.16 -1.23; 0.115) | 0.19 (0.04 -0.84; 0.029*) ⁿ |
| Escitalopram | 12 / 42 28.6) | 0.51 (0.29 -0.90; 0.021*) | 0.44 (0.25 -0.78; 0.005*) | 0.46 (0.27 -0.80; 0.006*) | 0.30 (0.28 -1.00; 0.050) ° | 40 / 84 (47.6) | 0.42 (0.22 -0.80; 0.008*) | 0.27 (0.13 -0.56; <0.001*) ^{p} |
| Fluoxetine | 4 / 14 (28.6) | 0.45 (0.17 -1.19; 0.108) | 0.30 (0.08 -1.17; 0.082) | 0.34 (0.11 -1.05; 0.062) | NA | 16 / 28 (57.1) | 0.24 (0.08 -0.73; 0.013*) | 0.18 (0.05 -0.70; 0.013*) |
| Paroxetine | 16 / 41 (39.0) | 0.69 (0.36 -1.33; 0.262) | 0.66 (0.38 -1.13; 0.130) | 0.57 (0.33 -0.98; 0.041*) | 0.62 (0.40 -0.97; 0.036*) ^{q} | 42 / 82 (51.2) | 0.54 (0.31 -0.94; 0.030*) | 0.48 (0.26 -0.89; 0.028* |
| Sertraline | 7/21 (33.3) | 0.58 (0.27 -1.21; 0.147) | 0.57 (0.29 -1.12; 0.110) | 0.63 (0.23 -1.69; 0.357) | 0.82 (0.34 -1.98; 0.663) ^{s} | 24 / 42 (57.1) | 0.42 (0.18 -0.98; 0.043*) | 0.42 (0.19 -0.91; 0.028* |
| Hydroxyzine | 11 / 31 (35.5) | 0.60 (0.33 -1.08; 0.090) | 0.43 (0.19 -0.96; 0.040*) | 0.46 (0.26 -0.84; 0.012*) | NA | 29 / 62 (46.8) | 0.49 (0.24 -0.99; 0.047*) | 0.46 (0.22 -0.99; 0.046*) ^{u} |
| FIASMA nervous system: psycholeptic | 4 / 13 (30.8) | 0.68 (0.28 -1.64; 0.387) | 0.59 (0.26 -1.35; 0.210) | NA | NA | 8/26 (30.8) | 0.82 (0.25 -2.73; 0.746) | 0.80 (0.21 -2.98; 0.735) ^{v} |
| Aripiprazole | 1 / 6 (16.7) | 0.27 (0.04 -1.95; 0.197) | NA | NA | NA | NA | NA | NA |
| Chlorpromazine | 3/7 (42.9) | 1.09 (0.48 -2.52; 0.829) | NA | NA | NA | NA | NA | NA |
| FIASMA respiratory system | 3/7 (42.9) | 1.02 (0.33 -3.18; 0.970) | 0.68 (0.25 -1.81; 0.439) | NA | NA | 7 / 14 (50.0) | 0.84 (0.22 -3.26; 0.798) | NA |
| Desloratadine | 3/7 (42.9) | 1.02 (0.33 -3.18; 0.970) | 0.68 (0.25 -1.81; 0.439) | NA | NA | 7 / 14 (50.0) | 0.84 (0.22 -3.26; 0.798) | NA |

^{a} Adjusted for age, cardiovascular disorders and diseases of the genitourinary system.
^{b} Adjusted for hospital, smoking, medication prescribed as part of a clinical trial or according to compassionate use, cardiovascular disorders, respiratory disorders and diseases of the genitourinary system.
^{c} Adjusted for cardiovascular disorders and diseases of the genitourinary system and endocrine disorders.
^{d} Adjusted for hospital, smoking, for diseases of the nervous system, respiratory disorders and diseases of the genitourinary system.
^{e} Adjusted for cardiovascular disorders and diseases of the genitourinary system and endocrine disorders.
^{f} Adjusted for hospital, smoking, for diseases of the nervous system, respiratory disorders and diseases of the genitourinary system.
^{g} Adjusted for hospital, obesity, smoking, medication prescribed as part of a clinical trial or according to compassionate use, respiratory disorders and diseases of the genitourinary system.
^{h} Adjusted for hospital, obesity, smoking, medication prescribed as part of a clinical trial or according to compassionate use, respiratory disorders and diseases of the genitourinary system.
ⁱ Adjusted for hospital.
^{j} Adjusted for age, sex, smoking and mental disorders.
^{k} Adjusted for hospital.
^{l} Adjusted for age, sex, smoking and mental disorders.
^{m} Adjusted for age, sex, hospital, smoking, medication prescribed as part of a clinical trial or according to compassionate use, neoplasms and diseases of the blood, diseases of the nervous system, digestive disorders and endocrine disorders.
ⁿ Adjusted for age, sex, hospital, other infectious diseases, cardiovascular disorders, respiratory disorders and endocrine disorders.
^{o} Adjusted for age, sex, hospital, obesity, smoking, medication prescribed as part of a clinical trial or according to compassionate use, other infectious diseases, neoplasms and diseases of the blood, cardiovascular disorders, respiratory disorders, digestive disorders, diseases of the genitourinary system, endocrine disorders and eye-ear-nose-throat disorders.
^{p} Adjusted for age, sex, hospital, obesity, smoking, medication prescribed as part of a clinical trial or according to compassionate use, neoplasms and diseases of the blood, digestive disorders and diseases of the genitourinary system.
^{q} Adjusted for age, hospital, obesity, other infectious diseases, mental disorders, diseases of the nervous system, digestive disorders, diseases of the musculoskeletal system, genitourinary system and endocrine disorders.
^{r} Adjusted for age, hospital, obesity, other infectious diseases, cardiovascular disorders, digestive disorders and endocrine disorders.
^{s} Adjusted for age, sex, hospital, obesity, smoking, medication prescribed as part of a clinical trial or according to compassionate use, other infectious diseases, neoplasms and diseases of the blood, mental disorders, diseases of the nervous system, digestive disorders, diseases of the musculoskeletal system, diseases of the genitourinary system, endocrine disorders and eye-ear-nose-throat disorders.
^{t} Adjusted for age, sex, hospital, smoking, medication prescribed as part of a clinical trial or according to compassionate use, other infectious diseases, mental disorders, diseases of the nervous system, respiratory disorders and diseases of the genitourinary system.
^{u} Adjusted for age, hospital, smoking, other infectious diseases, neoplasms and diseases of the blood, diseases of the nervous system, cardiovascular disorders, respiratory disorders and endocrine disorders.
^{v} Adjusted for age, sex, hospital, obesity, smoking, medication prescribed as part of a clinical trial or according to compassionate use, mental disorders and endocrine disorders.
*Two-sided p-value is significant (p<0.05).
Abbreviation: NA, not applicable.

TABLE 6

Association between FIASMA medication use at baseline and the endpoint of intubation or death as compared with paracetamol use at baseline among patients with mental disorder hospitalized for severe COVID-19.

| | Number of events / Number of patients | Crude Cox regression analysis | Multivariable Cox regression analysis | Analysis weighted by inverse-probability-weighting weights | Analysis weighted by inverse-probability-weighting weights adjusted for unbalanced covariates | Number of events / Number of patients in the matched groups | Univariate Cox regression in a 1:2 ratio matched analytic sample | Cox regression in a 1:2 ratio matched analytic sample^{a} adjusted for unbalanced covariates |
|---|---|---|---|---|---|---|---|---|
| | N (%) | HR (95CI; p-value) | HR (95CI; p-value) | HR (95CI; p-value) | HR (95CI; p-value) | N (%) | HR (95CI; p-value) | HR (95CI; p-value) |
| Any FIASMA medication | 104/277 (37.5) | 0.85 (0.65 -1.12; 0.242) | 0.69 (0.52 -0.92; 0.010*) | 0.63 (0.47 -0.86; 0.003*) | 0.65 (0.48 -0.88; 0.005*)° | 104/277 (37.5) | 0.72 (0.55 -0.94; 0.018*) | 0.70 (0.52 -0.94; 0.016*)^{p} |
| Paracetamol | 192 / 525 (36.6) | Ref. | Ref. | Ref. | Ref. | 110 / 277 (39.7) | Ref. | Ref. |
| FIASMA Alimentary tract and metabolism | 2 / 9 (22.2) | 0.41 (0.10 -1.69; 0.218) | 0.12 (0.01 -1.40; 0.091) | 0.35 (0.08 -1.49; 0.157) | 0.26 (0.13 -0.56; <0.001*)^{a} | 2 / 9 (22.2) | 0.37 (0.08 -1.76; 0.213) | NA |
| Paracetamol | 192 / 525 (36.6) | Ref. | Ref. | Ref. | Ref. | 8/18 (44.4) | Ref. | Ref. |
| FIASMA cardiovascular system | 54/125 (43.2) | 1.22 (0.83 -1.79; 0.319) | 0.76 (0.52 -1.10; 0.141) | 0.74 (0.40 -1.36; 0.331) | 0.71 (0.39 -1.28; 0.253) ^{b} | 54/125 (43.2) | 0.86 (0.62 -1.21; 0.397) | 0.73 (0.51 -1.03; 0.074) ^{c} |
| Paracetamol | 192 / 525 (36.6) | Ref. | Ref. | Ref. | Ref. | 71 / 250 (46.8) | Ref. | Ref. |
| FIASMA cardiovascular system: calcium channel blockers | 38 / 97 (39.2) | 0.86 (0.61 -1.22; 0.392) | 0.73 (0.50 -1.06; 0.098) | 0.60 (0.39 -0.93; 0.021*) | 0.64 (0.42 -0.97; 0.037*) ^{d} | 38 / 97 (39.2) | 0.67 (0.46 -0.98; 0.039*) | 0.67 (0.45 -1.00; 0.048*) ^{e} |
| Paracetamol | 192 / 525 (36.6) | Ref. | Ref. | Ref. | Ref. | 91/194 (46.9) | Ref. | Ref. |
| FIASMA cardiovascular system: other | 19 / 34 (55.9) | 1.88 (1.33 -2.67; <0.001*) | 1.02 (0.66 -1.60; 0.918) | 0.92 (0.53 -1.59; 0.754) | 1.19 (0.66 -2.13; 0.563) ^{f} | 19 / 34 (55.9) | 0.74 (0.41 -1.34; 0.322) | 0.78 (0.38 1.59; 0.486 |
| Paracetamol | 192 / 525 (36.6) | Ref. | Ref. | Ref. | Ref. | 33 / 68 (48.5) | Ref. | Ref. |
| FIASMA nervous system | 61/175 (34.9) | 0.77 (0.55 -1.07; 0.116) | 0.65 (0.46 -0.91; 0.013*) | 0.55 (0.36 -0.83; 0.004*) | 0.53 (0.36 -0.78; 0.001*) ^{h} | 61/175 (34.9) | 0.72 (0.52 -1.00; 0.047*) | 0.63 (0.45 0.89; 0.008*) ⁱ |
| Paracetamol | 192 / 525 (36.6) | Ref. | Ref. | Ref. | Ref. | 136 / 350 (38.9) | Ref. | Ref. |
| FIASMA nervous system: psychoanaleptic | 59 / 169 (34.9) | 0.77 (0.55 -1.07; 0.120) | 0.64 (0.45 -0.91; 0.013*) | 0.55 (0.38 -0.80; 0.002*) | 0.63 (0.45 -0.87; 0.005*)^{j} | 59 / 169 (34.9) | 0.70 (0.51 -0.97; 0.031*) | 0.53 (0.37 -0.74; <0.001*) ^{k} |
| Paracetamol | 192 / 525 (36.6) | Ref. | Ref. | Ref. | Ref. | 134/338 (39.6) | Ref. | Ref. |
| FIASMA nervous system: psycholeptic | 4 / 13 (30.8) | 0.59 (0.22 -1.58; 0.290) | 0.41 (0.13 -1.27; 0.122) | 0.35 (0.07 -1.72; 0.194) | 0.33 (0.12 -0.87; 0.026*) ^{l} | 4/13 (30.8) | 0.54 (0.18 -1.67; 0.288) | 0.16 (0.00 -10.45; 0.389) ^{m} |
| Paracetamol | 192 / 525 (36.6) | Ref. | Ref. | Ref. | Ref. | 13 / 26 (50.0) | Ref. | Ref. |
| FIASMA respiratory system | 3 / 7 (42.9) | 1.13 (0.36 -3.53; 0.835) | 0.97 (0.33 -2.89; 0.962) | 0.87 (0.25 -3.06; 0.830) | 0.19 (0.04 -0.95; 0.043*) ⁿ | 3 / 7 (42.9) | 2.23 (0.45 -11.14; 0.328) | NA |
| Paracetamol | 192 / 525 (36.6) | Ref. | Ref. | Ref. | Ref. | 3/14 (21.4) | Ref. | Ref. |

^{a} Adjusted for age, sex, obesity, smoking, medication according to compassionate use or as part of a clinical trial, other infectious diseases, mental disorders, respiratory disorders, digestive disorders, diseases of the musculoskeletal system, diseases of the genitourinary system and endocrine disorders.
^{b} Adjusted for hospital, medication according to compassionate use or as part of a clinical trial and other infectious diseases.
^{c} Adjusted for age_4g, hospital, obesity, smoking, other infectious diseases, mental disorders, cardiovascular disorders, respiratory disorders, digestive disorders, diseases of the genitourinary system and endocrine disorders.
^{d} Adjusted for hospital, smoking, other infectious diseases, diseases of the musculoskeletal system and endocrine disorders.
^{e} Adjusted for hospital, obesity, smoking, other infectious diseases, mental disorders, cardiovascular disorders, respiratory disorders, digestive disorders, diseases of the genitourinary system and endocrine disorders.
^{f} Adjusted for age, hospital, mental disorders, cardiovascular disorders, digestive disorders and diseases of the genitourinary system.
^{g} Adjusted for age, sex, hospital, obesity, smoking, medication according to compassionate use or as part of a clinical trial, neoplasms and diseases of the blood, mental disorders, cardiovascular disorders, respiratory disorders, digestive disorders, diseases of the musculoskeletal system, diseases of the genitourinary system and endocrine disorders.
^{h} Adjusted for age, hospital and dermatological disorders.
^{I} Adjusted for age, sex, hospital, obesity, smoking, medication according to compassionate use or as part of a clinical trial, other infectious diseases, neoplasms and diseases of the blood, mental disorders, diseases of the nervous system, cardiovascular disorders, digestive disorders, dermatological disorders, diseases of the genitourinary system and endocrine disorders and eye-ear-nose-throat disorders.
^{j} Adjusted for age, sex, medication according to compassionate use or as part of a clinical trial, mental disorders, diseases of the nervous system and dermatological disorders.
^{k} Adjusted for age, sex, obesity, smoking, medication according to compassionate use or as part of a clinical trial, other infectious diseases, neoplasms and diseases of the blood, mental disorders, diseases of the nervous system, cardiovascular disorders, digestive disorders, dermatological disorders, diseases of the genitourinary system and endocrine disorders.
^{l} Adjusted for age, sex, hospital, obesity, other infectious diseases, mental disorders, diseases of the nervous system, respiratory disorders, digestive disorders, diseases of the genitourinary system, endocrine disorders and eye-ear-nose-throat disorders.
^{m} Adjusted for age, sex, hospital, obesity, other infectious diseases, neoplasms and diseases of the blood, mental disorders, diseases of the nervous system, cardiovascular disorders, respiratory disorders and digestive disorders.
ⁿ Adjusted for age, sex, hospital, obesity, medication according to compassionate use or as part of a clinical trial, other infectious diseases, diseases of the nervous system, diseases of the genitourinary system and endocrine disorders.
^{o} Adjusted by age, any medication according to compassionate use or as part of a clinical trial, other infectious diseases, diseases of the nervous system, cardiovascular disorders, and diseases of the genitourinary system
^{p} Adjusted for age, sex, hospital, smoking, medication according to compassionate use or as part of a clinical trial, other infectious diseases, mental disorders, digestive disorders, diseases of the genitourinary system and endocrine disorders.
*Two-sided p-value is significant (p<0.05).
Abbreviation: NA, not applicable.

### 3. Discussion

In this multicenter retrospective observational study involving a relatively large sample of patients hospitalized for severe COVID-19 (N=2,846), we found that FIASMA medication use at study baseline was significantly and substantially associated with reduced risk of intubation or death, independently of sociodemographic characteristics and medical comorbidity. This association remained significant in multiple sensitivity analyses. Secondary exploratory analyses suggested that this association was not specific to one FIASMA class or medication in this population. These results suggest that the acid sphingomyelinase (ASM)/ceramide system may provide a useful framework for the repurposing of FIASMA medications against COVID-19 among individuals with severe COVID-19.

### Example 3

In this example part was examined the association between hydroxyzine use and the risk of death among adult patients who have been admitted to these medical centers with COVID-19, in time-to-event analyses adjusting for potential confounders, including patients’ characteristics (sex, age, obesity, current smoking status, number of medical conditions associated with increased COVID-19-related mortality, any medication prescribed according to compassionate use or as part of a clinical trial, and the presence of current sleep or anxiety disorder), disease’s severity at hospital admission (using markers of clinical and biological severity of COVID-19), and other psychotropic medications. Indeed, prior works have suggested that they may influence disease prognosis (including any benzodiazepine or Z-drug, any selective serotonin reuptake inhibitors (SSRI) antidepressant, any non-SSRI antidepressant, any mood stabilizer, and any antipsychotic medication). It was hypothesized that hydroxyzine use would be associated with reduced risk of death.

### 1. Material and Methods

### 1.1. Setting

This study was conducted at AP-HP, which comprises 39 hospitals, 23 of which are acute, 20 adult and 3 pediatric hospitals. Were included all adults aged 18 years or over who have been admitted with COVID-19 to these medical centers from the beginning of the epidemic in France, i.e. January 24th, until April 1st. COVID-19 was ascertained by a positive reverse-transcriptase-polymerase-chain-reaction (RT-PCR) test from analysis of nasopharyngeal or oropharyngeal swab specimens.

### 1.2. Data Sources

The data from the AP-HP Health Data Warehouse (‘Entrepôt de Données de Santé (EDS)’) were used. This anonymized warehouse contains all the clinical data available on all inpatient visits for COVID-19 to any of the 39 Greater Paris University hospitals. The data obtained included patients’ demographic characteristics, vital signs, laboratory test and RT-PCR test results, medication administration data, current medication lists, current diagnoses, and death certificates.

### 1.3. Variables Assessed

The following data were assessed for each patient at the time of the hospitalization: sex; age, which was categorized based on the OpenSAFELY study results (i.e. 18-50, 51-70, 71-80, 81+); obesity, defined as having a body-mass index higher than 30 kg/m² or an International Statistical Classification of Diseases and Related Health Problems (ICD-10) diagnosis code for obesity (E66.0, E66.1, E66.2, E66.8, E66.9); self-reported current smoking status; any medical condition associated with increased COVID-19-related mortality based on ICD-10 diagnosis codes, including diabetes mellitus (E11), diseases of the circulatory system (I00-I99), diseases of the respiratory system (J00-J99), neoplasms (C00-D49), diseases of the blood and blood-forming organs and certain disorders involving the immune mechanism (D5-D8), delirium (F05, R41), and dementia (G30, G31, F01-F03); any sleep or anxiety disorder (G47*, F40-F48); any medication prescribed according to compassionate use or as part of a clinical trial (e.g. hydroxychloroquine, azithromycin, remdesivir, tocilizumab, sarilumab or dexamethasone); clinical severity of COVID-19 at admission, defined as having at least one of the following criteria: respiratory rate > 24 breaths/min or < 12 breaths/min, resting peripheral capillary oxygen saturation in ambient air < 90%, temperature > 40° C., or systolic blood pressure < 100 mm Hg; and biological severity of COVID-19 at admission, defined as having at least one of the following criteria: high neutrophil-to-lymphocyte ratio or low lymphocyte-to-C-reactive protein ratio (both variables were dichotomized at the median of the values observed in the full sample), or plasma lactate levels higher than 2 mmol/L; any prescribed psychotropic medication, including any benzodiazepine or Z-drug, any selective serotonin reuptake inhibitors (SSRI) antidepressant, any non-SSRI antidepressant, any mood stabilizer (i.e. lithium or antiepileptic medications with mood stabilizing effects), and any antipsychotic medication.

All medical notes and prescriptions are computerized in Greater Paris University hospitals. Medications and their mode of administration (i.e., dosage, frequency, date, condition of intake) were identified from medication administration data or scanned handwritten medical prescriptions, through two deep learning models based on BERT contextual embeddings, one for the medications and another for their mode of administration. The model was trained on the APmed corpus, a previously annotated dataset for this task. Extracted medications names were then normalized to the Anatomical Therapeutic Chemical (ATC) terminology using approximate string matching.

### 1.4. Hydroxyzine Use

Study baseline was defined as the date of hospital admission. Hydroxyzine use was defined as receiving this medication at any time during the follow-up period, from study baseline to the end of the hospitalization or death.

### 1.5. Endpoint

The endpoint was the time from study baseline to death. Patients without an end-point event had their data censored on May 20th, 2020.

### 1.6. Statistical Analysis

frequencies and means (± standard deviations (SD)) of each baseline characteristic described above were calculated in patients receiving or not receiving hydroxyzine and compared them using chi-square tests or Welch’s t-tests.

To examine the associations between the use of hydroxyzine and the endpoint, Cox proportional-hazards regression models were performed. To help account for the nonrandomized prescription of hydroxyzine and reduce the effects of confounding, the primary analysis used propensity score analysis with inverse probability weighting. The individual propensities for receiving hydroxyzine were estimated using a multivariable logistic regression model that included patients’ characteristics (sex, age, obesity, current smoking status, number of medical conditions associated with increased COVID-19-related mortality, the presence of current sleep or anxiety disorder, any medication prescribed according to compassionate use or as part of a clinical trial), disease’s severity at hospital admission (using markers of clinical and biological severity of COVID-19), and other psychotropic medications (any benzodiazepine or Z-drug, any selective serotonin reuptake inhibitors (SSRI) antidepressant, any non-SSRI antidepressant, any mood stabilizer, and any antipsychotic medication). In the inverse-probability-weighted analyses, the predicted probabilities from the propensity-score models were used to calculate the stabilized inverse-probability-weighting weights. Associations between hydroxyzine use and the endpoint were then estimated using a multivariable Cox regression model including the inverse-probability-weighting weights. Kaplan-Meier curves were performed using the inverse-probability-weighting weights, and their pointwise 95% confidence intervals were estimated using the nonparametric bootstrap method.

Two sensitivity analyses were conducted, including a multivariable Cox regression model comprising as covariates the same variables as the inverse-probability-weighted analyses, and an univariate Cox regression model in a matched analytic samples using a 1:1 ratio, based on the same variables used for both the inverse-probability-weighted and the multivariable Cox regression analyses. Weighted Cox regression models were used when proportional hazards assumption was not met. To reduce the effects of confounding, optimal matching was used in order to obtain the smallest average absolute distance across all clinical characteristics between exposed patient and non-exposed matched controls.

Finally, within patients receiving hydroxyzine, the association of cumulative dose received (dichotomized by the median dose) with the endpoint was tested.

For all associations, residual analyses were performed to assess the fit of the data, check assumptions, including proportional hazards assumptions, and examined the potential influence of outliers. Statistical significance was fixed a priori at two-sided p-value<0.05. All analyses were conducted in R software version 2.4.3 (R Project for Statistical Computing).

### 2. Results

### 2.1. Characteristics of the Cohort

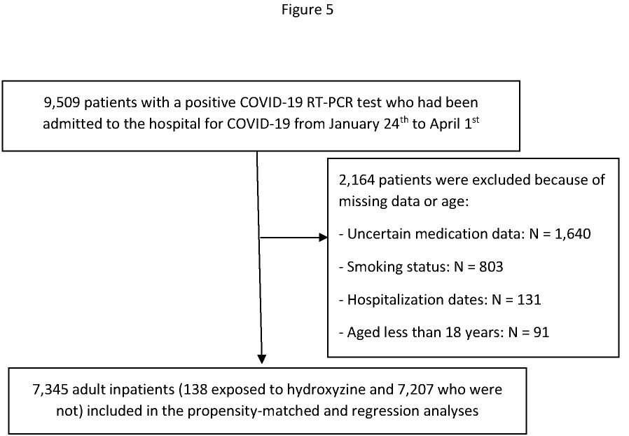

Of the 9,509 patients with a positive COVID-19 RT-PCR test consecutively admitted to the hospital, a total of 2,164 patients (22.8%) were excluded because of missing data (outside clinical and biological markers of severity) or their young age (i.e. less than 18 years old of age). Of the remaining 7,345 adult inpatients, 138 (1.9%) patients received hydroxyzine during the hospitalization, at a median daily dose of 25 mg (mean=49.8 mg, SD=51.5, minimum=12.5, maximum=300.0) per day (FIG. 5).

COVID-19 RT-PCR test results were obtained after a mean delay of 5 days (SD=11.7, median=1 day) from the date of hospital admission. This delay was not significantly different between patients receiving or not receiving hydroxyzine [mean delay in the exposed group=7.1 day (SD=14.9); mean delay in the non-exposed group=5.0 day (SD=11.7); Welch’s t-test=-1.63, p=0.106)].

Over a mean follow-up of 20.3 days (SD=27.5; median=7 days; range: 1 day to 117 days), 994 patients (13.5%) had an end-point event at the time of data cutoff on May 20th. Among patients receiving hydroxyzine, the mean follow-up was 22.4 days (SD=25.9; median=12.5 days; range: 1 day to 114 days), while it was of 20.2 days (SD=27.5; median=6 days; range: 4 day to 117 days) in those who were not (Welch’s t-test=-0.97, p=0.336).

All baseline characteristics were independently and significantly associated with mortality, except for current smoking, any current sleep or anxiety disorder, any non-SSRI antidepressant, any mood stabilizer, and any antipsychotic medication (Table 7).

TABLE 7

Associations of baseline clinical characteristics with the endpoint of death in the cohort of patients who had been admitted to the hospital for Covid-19 (N=7,345).

| | | | | Endpoint of death | | |
|---|---|---|---|---|---|---|
| | | With the end-point event (N=994) | Without the end-point event (N=6,351) | Crude analysis | Mutivariable analysis | |
| | N (%) | N (%) | N (%) | HR (SE) / p-value | HR (SE) / p-value | Collinearity diagnosis (VIF) |
| Age | | | | | | 1.06 |
| 18 to 50 years | 2709 (36.9%) | 42 (4.23%) | 2667 (42.0%) | Ref. | Ref. | |
| 51 to 70 years | 2530 (34.4%) | 240 (24.1%) | 2290 (36.1%) | 5.31 (0.17) / <0.001* | 2.80 (0.19) / <0.001* | |
| 71 to 80 years | 942 (12.8%) | 273 (27.5%) | 669 (10.5%) | 14.86 (0.17) / <0.001* | 7.51 (0.20) / <0.001* | |
| More than 80 years | 1164 (15.8%) | 439 (44.2%) | 725 (11.4%) | 16.99 (0.16) / <0.001* | 11.30 (0.19) / <0.001* | |
| Sex | | | | | | 1.06 |
| Women | 3619 (49.3%) | 359 (36.1%) | 3260 (51.3%) | Ref. | | |
| Men | 3726 (50.7%) | 635 (63.9%) | 3091 (48.7%) | 1.68 (0.07) / <0.001* | 1.32 (0.08) / <0.001* | |
| Obesity | | | | | | 1.06 |
| Yes | 975 (13.3%) | 216 (21.7%) | 759 (12.0%) | 1.51 (0.08) / <0.001* | 1.22 (0.09) / 0.024* | |
| No | 6370 (86.7%) | 778 (78.3%) | 5592 (88.0%) | Ref. | Ref. | |
| Smoking | | | | | | 1.03 |
| Yes | 623 (8.48%) | 149 (15.0%) | 474 (7.46%) | 1.51 (0.09) / <0.001* | 0.93 (0.10) / 0.447 | |
| No | 6722 (91.5%) | 845 (85.0%) | 5877 (92.5%) | Ref. | Ref. | |
| Any Medical Condition | | | | | | 1.12 |
| 0 | 4750 (64.7%) | 319 (32.1%) | 4431 (69.8%) | Ref. | Ref. | |
| 1 | 534 (7.27%) | 63 (6.34%) | 471 (7.42%) | 2.29 (0.14) / <0.001* | 1.78 (0.16) / <0.001* | |
| 2 or more | 2061 (28.1%) | 612 (61.6%) | 1449 (22.8%) | 4.71 (0.07) / <0.001* | 3.50 (0.09) / <0.001* | |
| Medication according to compassionate use or as part of a clinical trial | | | | | | 1.11 |
| Yes | 1288 (17.5%) | 226 (22.7%) | 1062 (16.7%) | 1.13 (0.08) / 0.105 | 0.73 (0.09) / <0.001* | |
| No | 6057 (82.5%) | 768 (77.3%) | 5289 (83.3%) | Ref. | Ref. | |
| Anxiety or insomnia | | | | | | 1.04 |
| Yes | 299 (4.07%) | 101 (10.2%) | 198 (3.12%) | 2.38 (0.11) / <0.001* | 1.24 (0.12) / 0.067 | |
| No | 7046 (95.9%) | 893 (89.8%) | 6153 (96.9%) | Ref. | Ref. | |
| SSRI or SNRI | | | | | | 1.07 |
| Yes | 257 (3.50%) | 68 (6.84%) | 189 (2.98%) | 1.53 (0.13) / 0.001* | 0.65 (0.13) / 0.001* | |
| No | 7088 (96.5%) | 926 (93.2%) | 6162 (97.0%) | Ref. | Ref. | |
| Any other antidepressant | | | | | | 1.06 |
| Yes | 203 (2.76%) | 60 (6.04%) | 143 (2.25%) | 1.84 (0.13) / <0.001* | 0.83 (0.15) / 0.199 | |
| No | 7142 (97.2%) | 934 (94.0%) | 6208 (97.7%) | Ref. | Ref. | |
| Any mood stabilizer medication | | | | | | 1.05 |
| Yes | 287 (3.91%) | 69 (6.94%) | 218 (3.43%) | 1.42 (0.12) / 0.005* | 0.90 (0.14) / 0.444 | |
| No | 7058 (96.1%) | 925 (93.1%) | 6133 (96.6%) | Ref. | Ref. | |
| Any benzodiazepine or Z-drug | | | | | | 1.14 |
| Yes | 752 (10.2%) | 268 (27.0%) | 484 (7.62%) | 2.53 (0.07) / <0.001* | 1.56 (0.09) / <0.001* | |
| No | 6593 (89.8%) | 726 (73.0%) | 5867 (92.4%) | Ref. | Ref. | |
| Any antipsychotic | | | | | | 1.07 |
| Yes | 264 (3.59%) | 60 (6.04%) | 204 (3.21%) | 1.26 (0.13) / 0.086 | 0.81 (0.14) / 0.131 | |
| No | 7081 (96.4%) | 934 (94.0%) | 6147 (96.8%) | Ref. | Ref. | |
| Cinical severity of Covid-19 at admission | | | | | | 1.15 |
| Yes | 1564 (21.3%) | 421 (42.4%) | 1143 (18.0%) | 1.81 (0.08) / <0.001* | 1.69 (0.09) / <0.001* | |
| No | 1858 (25.3%) | 265 (26.7%) | 1593 (25.1%) | Ref. | Ref. | |
| Missing | 3923 (53.4%) | 308 (31.0%) | 3615 (56.9%) | 0.59 (0.08) / <0.001* | 1.66 (0.11) / <0.001* | |
| Biological severity of Covid-19 at admission | | | | | | 1.15 |
| Yes | 2439 (33.2%) | 592 (59.6%) | 1847 (29.1%) | 1.94 (0.08) / <0.001* | 1.50 (0.09) / <0.001* | |
| No | 1861 (25.3%) | 257 (25.9%) | 1604 (25.3%) | Ref. | Ref. | |
| Missing | 3045 (41.5%) | 145 (14.6%) | 2900 (45.7%) | 0.39 (0.1) / <0.001* | 0.75 (0.12) / <0.020* | |

^{α} Defined as having a body-mass index higher than 30 kg/m² or an International Statistical Classification of Diseases and Related Health Problems (ICD-10) diagnosis code for obesity (E66.0, E66.1, E66.2, E66.8, E66.9).
^{β} Smoking status was self-reported.
^{γ} Assessed using ICD-10 diagnosis codes for diabetes mellitus (E11), diseases of the circulatory system (I00-I99), diseases of the respiratory system (J00-J99), neoplasms (C00-D49), diseases of the blood and blood-forming organs and certain disorders involving the immune mechanism (D5-D8), delirium (F05, R41) and dementia (G30, G31, F01-F03).
^{Θ} Any medication prescribed as part of a clinical trial or according to compassionate use (e.g., hydroxychloroquine, azithromycin, remdesivir, tocilizumab, sarilumab or dexamethasone).
^{€} Assessed using ICD-10 diagnosis codes for anxiety, dissociative, stress-related, somatoform and other nonpsychotic mental disorders (F40-F48) and insomnia (G47).
^{Ω} Included lithium or antiepileptic medications with mood stabilizing properties.
^{µ} Defined as having at least one of the following criteria: respiratory rate > 24 breaths/min or < 12 breaths/min, resting peripheral capillary oxygen saturation in ambient air < 90%, temperature > 40° C., or systolic blood pressure < 100 mm Hg.
^{κ} Defined as having at least one of the following criteria: high neutrophil-to-lymphocyte ratio, low lymphocyte-to-C-reactive protein (both variables were dichotomized at the median of the values observed in the full sample), and plasma lactate levels higher than 2 mmol/L.
*p-value is significant (p<0.05).
Abbreviations: HR, hazard ratio; SE, standard error; VIF, variance inflation factor.

The distribution of patients’ characteristics according to hydroxyzine use is shown in Table 8. In the full sample, hydroxyzine use significantly differed according to all baseline characteristics (Table 8). The direction of these associations indicated older age and overall greater medical severity of patients receiving hydroxyzine than those who did not. After applying the propensity score weights, these differences were substantially reduced (Table 8). In the matched analytic sample, there were no significant differences in patients’ characteristics according to hydroxyzine use (Table 8).

TABLE 8

Characteristics of patients with COVID-19 receiving or not receiving hydroxyzine.

| | Exposed to hydroxyzine (N=138) | Not exposed to hydroxyzine (N=7,207) | Non-exposed matched group (N=138) | Exposed to hydroxyzine vs. Not exposed to hydroxyzine | Exposed to hydroxyzine vs. Not exposed to hydroxyzine | Exposed to hydroxyzine vs. Non-exposed matched group |
|---|---|---|---|---|---|---|
| | | | | Crude analysis | Analysis weighted by inverse-probability-weighting weights | Matched analytic sample analysis |
| | | | | Chi-square test | Weighted Chi-square test | Chi-square test |
| | N (%) | N (%) | N (%) | | | |
| | | | | (p-value) | (p-value) | (p-value) |
| Age | | | | 13.97 (0.003^{∗}) | 2.90 (0.408) | 1.13 (0.771) |
| 18 to 50 years | 35 (25.4%) | 2674 (37.1%) | 28 (20.3%) | | | |
| 51 to 70 years | 59 (42.8%) | 2471 (34.3%) | 61 (44.2%) | | | |
| 71 to 80 years | 27 (19.6%) | 915 (12.7%) | 29 (21.0%) | | | |
| More than 80 years | 17 (12.3%) | 1147 (15.9%) | 20 (14.5%) | | | |
| Sex | | | | 6.21 (0.013*) | 1.96 (0.162) | 0.02 (0.901) |
| Women | 53 (38.4%) | 3566 (49.5%) | 51 (37.0%) | | | |
| Men | 85 (61.6%) | 3641 (50.5%) | 87 (63.0%) | | | |
| Obesity | | | | 14.79 (<0.001*) | 5.25 (0.022*) | 0.52 (0.471) |
| Yes | 34 (24.6%) | 941 (13.1%) | 28 (20.3%) | | | |
| No | 104 (75.4%) | 6266 (86.9%) | 110 (79.7%) | | | |
| Smoking | | | | 11.09 (0.001*) | 3.67 (0.055) | 0.03 (0.869) |
| Yes | 23 (16.7%) | 600 (8.33%) | 21 (15.2%) | | | |
| No | 115 (83.3%) | 6607 (91.7%) | 117 (84.8%) | | | |
| Any medical condition | | | | 84.95 (<0.001*) | 33.78 (<0.001*) | 0.018 (0.912) |
| 0 | 40 (29.0%) | 4710 (65.4%) | 41 (29.7%) | | | |
| 1 | 13 (9.42%) | 521 (7.23%) | 11 (7.97%) | | | |
| 2 or more | 85 (61.6%) | 1976 (27.4%) | 86 (62.3%) | | | |
| Medication according to compassionate use or as part of a clinical trial | | | | 40.9 (<0.001*) | 10.94 (0.001*) | <0.01 (>0.99) |
| Yes | 53 (38.4%) | 1235 (17.1%) | 54 (39.1%) | | | |
| No | 85 (61.6%) | 5972 (82.9%) | 84 (60.9%) | | | |
| Anxiety of insomnia | | | | 26.7 (<0.001*) | 14.32 (<0.001*) | 0.03 (0.855) |
| Yes | 18 (13.0%) | 281 (3.90%) | 16 (11.6%) | | | |
| No | 120 (87.0%) | 6926 (96.1%) | 122 (88.4%) | | | |
| SSRIor SNRI | | | | 9.73 (0.002*) | 1.12 (0.290) | <0.01 (>0.99) |
| Yes | 12 (8.70%) | 245 (3.40%) | 12 (8.70%) | | | |
| No | 126 (91.3%) | 6962 (96.6%) | 126 (91.3%) | | | |
| Any other antidepressant | | | | 16.23 (<0.001*) | 2.45 (0.117) | <0.01 (>0.99) |
| Yes | 12 (8.70%) | 191 (2.65%) | 12 (8.70%) | | | |
| No | 126 (91.3%) | 7016 (97.3%) | 126 (91.3%) | | | |
| Any mood stabilizer medication | | | | 20.10 (<0.001*) | 5.16 (0.023*) | <0.01 (>0.99) |
| Yes | 16 (11.6%) | 271 (3.76%) | 16 (11.6%) | | | |
| No | 122 (88.4%) | 6936 (96.2%) | 122 (88.4%) | | | |
| Any benzodiazepine or Z-drug | | | | 69.32 (<0.001*) | 26.21 (<0.001*) | <0.01 (>0.99) |
| Yes | 44 (31.9%) | 708 (9.82%) | 45 (32.6%) | | | |
| No | 94 (68.1%) | 6499 (90.2%) | 93 (67.4%) | | | |
| Any antipsychotic | | | | 65.57 (<0.001*) | 24.29 (<0.001*) | <0.01 (>0.99) |
| Yes | 23 (16.7%) | 241 (3.34%) | 23 (16.7%) | | | |
| No | 115 (83.3%) | 6966 (96.7%) | 115 (83.3%) | | | |
| Clinical severity of Covid-19 at admission | | | | 48.7 (<0.001*) | 19.13 (<0.001*) | 0.17 (0.917) |
| Yes | 53 (38.4%) | 1511 (21.0%) | 50 (36.2%) | | | |
| No | 51 (37.0%) | 1807 (25.1%) | 54 (39.1%) | | | |
| Missing | 34 (24.6%) | 3889 (54.0%) | 34 (24.6%) | | | |
| Biological severity of Covid-19 at admission | | | | 29.65 (<0.001*) | 3.52 (0.172) | 0.94 (0.626) |
| Yes | 69 (50.0%) | 2370 (32.9%) | 74 (53.6%) | | | |
| No | 42 (30.4%) | 1819 (25.2%) | 43 (31.2%) | | | |
| Missing | 27 (19.6%) | 3018 (41.9%) | 21 (15.2%) | | | |

^{α} Defined as having a body-mass index higher than 30 kg/m² or an International Statistical Classification of Diseases and Related Health Problems (ICD-10) diagnosis code for obesity (E66.0, E66.1, E66.2, E66.8, E66.9).
^{β} Smoking status was self-reported.
^{γ} Assessed using ICD-10 diagnosis codes for diabetes mellitus (E11), diseases of the circulatory system (I00-I99), diseases of the respiratory system (J00-J99), neoplasms (C00-D49), diseases of the blood and blood-forming organs and certain disorders involving the immune mechanism (D5-D8), delirium (F05, R41) and dementia (G30, G31, F01-F03).
^{Θ} Any medication prescribed as part of a clinical trial or according to compassionate use (e.g., hydroxychloroquine, azithromycin, remdesivir, tocilizumab, sarilumab or dexamethasone).
^{€} Assessed using ICD-10 diagnosis codes for anxiety, dissociative, stress-related, somatoform and other nonpsychotic mental disorders (F40-F48) and insomnia (G47).
^{Ω} Included lithium or antiepileptic medications with mood stabilizing properties.
^{µ} Defined as having at least one of the following criteria: respiratory rate > 24 breaths/min or < 12 breaths/min, resting peripheral capillary oxygen saturation in ambient air < 90%, temperature > 40° C., or systolic blood pressure < 100 mm Hg.
^{κ} Defined as having at least one of the following criteria: high neutrophil-to-lymphocyte ratio, low lymphocyte-to-C-reactive protein (both variables were dichotomized at the median of the values observed in the full sample), and plasma lactate levels higher than 2 mmol/L.
*p-value is significant p<0.05).
Abbreviations: HR, hazard ratio; SE, standard error.

### 2.2. Study Endpoint

Among patients receiving hydroxyzine, death occurred in 15 patients (10.9%), while 979 non-exposed patients (16.6%) had this outcome (Table 9). Unadjusted hazard ratio of the association between hydroxyzine use and the endpoint of death was not significant (HR, 0.60, 95% CI, 0.36 to 1.00, p=0.051) (Table 9).

TABLE 9

Association between hydroxyzine use and the endpoint of death in the full sample and in the matched analytic sample

| | Number of events / Number of patients | Crude Cox regression analysis | Multivariable Cox regression analysis | Analysis weighted by inverse-probability-weighting weights | Number of events/ Number of patients | Univariate Cox regression in the matched analytic sample (1:1) |
|---|---|---|---|---|---|---|
| | N (%) | HR (95% CI; p-value) | HR (95% CI; p-value) | HR (95% CI; p-value) | N (%) | HR (95% CI; p-value) |
| No hydroxyzine | 979 / 7,207 (16.6%) | Ref. | Ref. | Ref. | 29/138 (21.0%) | Ref. |
| Hydroxyzine | 15 / 138 (10.9%) | 0.60 (0.36 - 1.00; 0.051) | 0.42 (0.25 - 0.71; 0.001*) | 0.40 (0.24 - 0.68; 0.001*) | 15 / 138 (10.9%) | 0.37 (0.20 - 0.69; 0.002*) |

*p-value is significant (p<0.05).

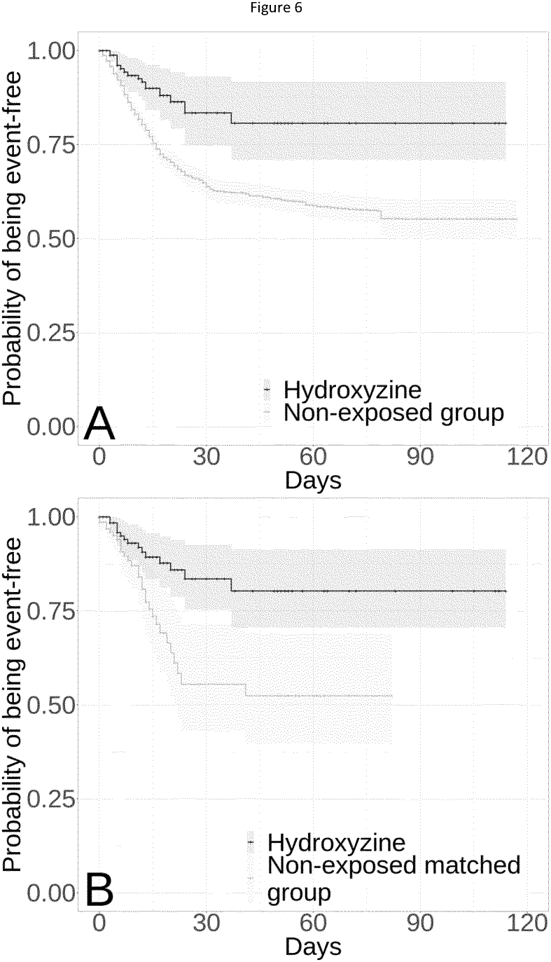

However, the primary multivariable analysis with inverse probability weighting taking into account differences in baseline characteristics showed a significant association between hydroxyzine use and reduced risk of death (HR, 0.40; 95% CI, 0.24 to 0.68, p=0.001) (Table 9, FIG. 6).

In sensitivity analyses, the multivariable Cox regression model in the full sample yielded similar results (HR, 0.42; 95% CI, 0.25 to 0.71, p=0.001), as did the univariate Cox regression model in the matched analytic sample (HR, 0.37; 95% CI, 0.20 to 0.69, p=0.002) (Table 9, FIG. 6).

Finally, exposure to higher (mean=2908.6 mg, median=1250 mg, SD=2317.2, median daily dose=75 mg, SD=63.6) rather than lower (mean=202.6, median=175 mg, SD=126.9, median daily dose=25 mg, SD=4.2) cumulative doses of hydroxyzine was significantly associated with lower risk of death (Table 10).

TABLE 10

Associations of hydroxyzine dose with the endpoint of death among patients using hydroxyzine

| | Number of events / Number of patients | Crude Cox regression analysis | Cox regression adjusted for age and sex | Multivariable Cox regression analysis |
|---|---|---|---|---|
| | N (%) | HR (95% CI; p-value) | HR (95% CI; p-value) | HR (95% CI; p-value) |
| Cumulative dose | | | | |
| Low doses | 8 / 53 (15.1%) | Ref. | Ref. | Ref. |
| High doses | 2 / 53 (3.8%) | 0.10 (0.02 - 0.48; 0.004*) | 0.12 (0.03 - 0.57; 0.007*) | 0.10 (0.02 - 0.45; 0.003*) |

*p-value is significant (p<0.05).
5 The variable cumulative dose has been dichotomized by the median.

A post-hoc analysis indicated that in the full sample, we had 80% power to detect hazard ratios for hydroxyzine of at least 1.93/0.32 and 2.54/0.18 for the endpoint of death in the full sample and in the matched analytic sample, respectively.

### 3. Discussion

In this multicenter retrospective observational study involving a large sample of patients admitted to the hospital with COVID-19, it was found that hydroxyzine use, at a median daily dose of 25 mg (mean 49.8 mg, SD=51.5), was significantly and substantially associated with reduced risk of death, independently of patients’ characteristics, clinical and biological markers of disease’s severity at hospital admission, and use of other psychotropic medications. That association was significantly stronger at higher rather than lower cumulative doses.

In the analyses, the effects of confounding were minimized in several different ways. First, a multivariable regression model was used, with inverse probability weighting to minimize the effects of confounding by indication. Sensitivity analyses were also performed, including a multivariable Cox regression models and an univariate Cox regression model in a matched analytic sample, that showed similar results. Second, although some amount of unmeasured confounding may remain, the present analyses adjusted for numerous potential confounders, including patients’ characteristics (sex, age, obesity, current smoking status, number of medical conditions associated with increased COVID-19-related mortality, any medication prescribed according to compassionate use or as part of a clinical trial, and the presence of current sleep or anxiety disorder), disease’s severity at hospital admission (using markers of clinical and biological severity of COVID-19), and other psychotropic medications as prior work have suggested that they may influence disease prognosis (including any benzodiazepine or Z-drug, any selective serotonin reuptake inhibitors (SSRI) antidepressant, any non-SSRI antidepressant, any mood stabilizer, and any antipsychotic medication).

In this multicenter observational retrospective study involving patients admitted to the hospital for COVID-19, hydroxyzine use during the hospitalization was significantly and substantially associated with lower risk of death, independently of patients’ characteristics, clinical and biological markers of disease’s severity at hospital admission, and use of other psychotropic medications.

### BIBLIOGRAPHIC REFERENCES

1. Hoertel N, Blachier M, Blanco C, Olfson M, Massetti M, Sanchez-Rico M, et al. A stochastic agent-based model of the SARS-CoV-2 epidemic in France. Nat Med. 2020;26:1417-1421.

2. Hoertel N, Blachier M, Blanco C, Olfson M, Massetti M, Limosin F, et al. Facing the COVID-19 epidemic in NYC: a stochastic agent-based model of various intervention strategies. MedRxiv. 2020. 2020. https://doi.org/10.1101/2020.04.23.20076885.

3. Chevance A, Gourion D, Hoertel N, Llorca P-M, Thomas P, Bocher R, et al. Ensuring mental health care during the SARS-CoV-2 epidemic in France: A narrative review. L′Encephale. 2020;46:193-201.

4. Carpinteiro A, Edwards MJ, Hoffmann M, Kochs G, Gripp B, Weigang S, et al. Pharmacological inhibition of acid sphingomyelinase prevents uptake of SARS-CoV-2 by epithelial cells. Cell Rep Med. 2020:100142.

5. Kornhuber J, Tripal P, Reichel M, Mühle C, Rhein C, Muehlbacher M, et al. Functional Inhibitors of Acid Sphingomyelinase (FIASMAs): a novel pharmacological group of drugs with broad clinical applications. Cell Physiol Biochem. 2010;26:9-20.

6. Schloer S, Brunotte L, Goretzko J, Mecate-Zambrano A, Korthals N, Gerke V, et al. Targeting the endolysosomal host-SARS-CoV-2 interface by clinically licensed functional inhibitors of acid sphingomyelinase (FIASMA) including the antidepressant fluoxetine. Emerg Microbes Infect. 2020;9:2245-2255.

7. Lenze EJ, Mattar C, Zorumski CF, Stevens A, Schweiger J, Nicol GE, et al. Fluvoxamine vs Placebo and Clinical Deterioration in Outpatients With Symptomatic COVID-19: A Randomized Clinical Trial. JAMA. 2020;324:2292-2300.

8. Hoertel N, Sánchez-Rico M, Vernet R, Beeker N, Jannot A-S, Neuraz A, et al. SSRIs and SNRIs and Risk of Death or Intubation in COVID-19: Results from an Observational Study. MedRxiv. 2020 (published on Jul. 14, 2020).

9. Hoertel N, Sánchez M, Vernet R, Beeker N, Neuraz A, Blanco C, et al. Association between Hydroxyzine Use and Reduced Mortality in Patients Hospitalized for Coronavirus Disease 2019: Results from a multicenter observational study. MedRxiv. 2020 (published on Oct. 27, 2020).

10. Cipriani A, Furukawa TA, Salanti G, Chaimani A, Atkinson LZ, Ogawa Y, et al. Comparative efficacy and acceptability of 21 antidepressant drugs for the acute treatment of adults with major depressive disorder: a systematic review and network meta-analysis. Focus. 2018; 16:420-429.

11. Leucht S, Corves C, Arbter D, Engel RR, Li C, Davis JM. Second-generation versus first-generation antipsychotic drugs for schizophrenia: a meta-analysis. The Lancet. 2009;373:31-41.

12. Gulbins E, Palmada M, Reichel M, Lüth A, Böhmer C, Amato D, et al. Acid sphingomyelinase-ceramide system mediates effects of antidepressant drugs. Nat Med. 2013;19:934-938.

13. Kornhuber J, Muehlbacher M, Trapp S, Pechmann S, Friedl A, Reichel M, et al. Identification of novel functional inhibitors of acid sphingomyelinase. PloS One. 2011;6:e23852.

14. Hoertel N, Rico MS, Vernet R, Jannot A-S, Neuraz A, Blanco C, et al. Observational Study of Haloperidol in Hospitalized Patients with Covid-19. MedRxiv. 2020

15. Hoertel N, Sanchez Rico M, Vernet R, Jannot A-S, Neuraz A, Blanco C, et al. Observational Study of Chlorpromazine in Hospitalized Patients with Covid-19. MedRxiv. 2020. 2020.

16. Hoertel N, Sánchez M, Vernet R, Beeker N, Neuraz A, Alvarado J, et al. Dexamethasone use and Mortality in Hospitalized Patients with Coronavirus Disease 2019: a Multicenter Retrospective Observational Study. MedRxiv. 2020. 2020.

17. Li L, Li F, Fortunati F, Krystal JH. Association of a Prior Psychiatric Diagnosis With Mortality Among Hospitalized Patients With Coronavirus Disease 2019 (COVID-19) Infection. JAMA Netw Open. 2020;3:e2023282-e2023282.

18. Wang QQ, Kaelber DC, Xu R, Volkow ND. COVID-19 risk and outcomes in patients with substance use disorders: analyses from electronic health records in the United States. Mol Psychiatry. 2020:1-10.

19. Haut Conseil de la Santé Publique. Statement on the management at home or in a care facility of suspected or confirmed Covid-19 patients. 2020.

20. Lagunas-Rangel FA. Neutrophil-to-lymphocyte ratio and lymphocyte-to-C-reactive protein ratio in patients with severe coronavirus disease 2019 (COVID-19): A meta-analysis. J Med Virol. 2020. 2020.

21. Zhang C, Wu Z, Li J-W, Zhao H, Wang G-Q. The cytokine release syndrome (CRS) of severe COVID-19 and interleukin-6 receptor (IL-6R) antagonist tocilizumab may be the key to reduce the mortality. Int J Antimicrob Agents. 2020;55.

22. Terry M. Therneau, Patricia M. Grambsch. Modeling Survival Data: Extending the Cox Model. New York: Springer; 2000.

23. Geleris J, Sun Y, Platt J, Zucker J, Baldwin M, Hripcsak G, et al. Observational Study of Hydroxychloroquine in Hospitalized Patients with Covid-19. N Engl J Med. 2020;382:2411-2418.

24. Robins JM, Hernán MÁ, Brumback B. Marginal Structural Models and Causal Inference in Epidemiology. Epidemiology. 2000; 11:550-560.

25. Venables WN, Ripley BD. Modern Applied Statistics with S. Fourth. New York: Springer; 2002.

26. Efron B. Nonparametric standard errors and confidence intervals. Can J Stat. 1981;9:139-158.

27. Kassambara A, Kosinski M, Biecek P. survminer: Drawing Survival Curves using ‘ggplot2’. 2020.

28. Hansen BB, Klopfer SO. Optimal full matching and related designs via network flows. J Comput Graph Stat. 2006;15:609-627.

29. Grambsch PM, Therneau TM. Proportional hazards tests and diagnostics based on weighted residuals. Biometrika. 1994;81:515-526.

30. Ishima T, Fujita Y, Hashimoto K. Interaction of new antidepressants with sigma-1 receptor chaperones and their potentiation of neurite outgrowth in PC12 cells. Eur J Pharmacol. 2014;727:167-173.

31. Ishikawa M, Ishiwata K, Ishii K, Kimura Y, Sakata M, Naganawa M, et al. High occupancy of sigma-1 receptors in the human brain after single oral administration of fluvoxamine: a positron emission tomography study using [11C] SA4503. Biol Psychiatry. 2007;62:878-883.

32. Zhou Y, Hou Y, Shen J, Huang Y, Martin W, Cheng F. Network-based drug repurposing for novel coronavirus 2019-nCoV/SARS-CoV-2. Cell Discov. 2020;6:1-18.

33. Hashimoto K. Activation of sigma-1 receptor chaperone in the treatment of neuropsychiatric diseases and its clinical implication. J Pharmacol Sci. 2015; 127:6-9.

34. Krutetskaya Z, Melnitskaya A, Antonov V, Nozdrachev A. Sigma-1 receptor antagonists haloperidol and chlorpromazine modulate the effect of glutoxim on NA+ transport in frog skin. Dokl. Biochem. Biophys., vol. 484, Springer; 2019. p. 63-65.

35. Vela JM. Repurposing sigma-1 receptor ligands for Covid-19 therapy? Front Pharmacol. 2020; 11: 1716.

36. Köhler CA, Freitas TH, Stubbs B, Maes M, Solmi M, Veronese N, et al. Peripheral alterations in cytokine and chemokine levels after antidepressant drug treatment for major depressive disorder: systematic review and meta-analysis. Mol Neurobiol. 2018;55:4195-4206.

37. Hojyo S, Uchida M, Tanaka K, Hasebe R, Tanaka Y, Murakami M, et al. How COVID-19 induces cytokine storm with high mortality. Inflamm Regen. 2020;40:1-7.

38. Ye Q, Wang B, Mao J. The pathogenesis and treatment of theCytokine Storm’in COVID-19. J Infect. 2020;80:607-613.

39. Obuchowicz E, Bielecka-Wajdman AM, Paul-Samojedny M, Nowacka M. Different influence of antipsychotics on the balance between pro-and anti-inflammatory cytokines depends on glia activation: an in vitro study. Cytokine. 2017;94:37-44.

40. Sugino H, Futamura T, Mitsumoto Y, Maeda K, Marunaka Y. Atypical antipsychotics suppress production of proinflammatory cytokines and up-regulate interleukin-10 in lipopolysaccharide-treated mice. Prog Neuropsychopharmacol Biol Psychiatry. 2009;33:303-307.

41. Mengozzi M, Fantuzzi G, Faggioni R, Marchant A, Goldman M, Orencole S, et al. Chlorpromazine specifically inhibits peripheral and brain TNF production, and up-regulates IL-10 production, in mice. Immunology. 1994;82:207.

43. Devlin J, Chang M-W, Lee K, Toutanova K. Bert: Pre-training of deep bidirectional transformers for language understanding. ArXiv. 2018. 2018. https://doi.org/1810.04805.

44. Jouffroy J, Feldman SF, Lerner I, Rance B, Neuraz A, Burgun A. MedExt: combining expert knowledge and deep learning for medication extraction from French clinical texts. 2020. 23 Jan. 2020.

45. Williamson E, Walker AJ, Bhaskaran KJ, Bacon S, Bates C, Morton CE, et al. OpenSAFELY: factors associated with COVID-19-related hospital death in the linked electronic health records of 17 million adult NHS patients. MedRxiv. 2020.

46. Devlin J, Chang M-W, Lee K, Toutanova K. Bert: Pre-training of deep bidirectional transformers for language understanding. ArXiv. 2018. 2018. https://doi.org/1810.04805.

47. Jouffroy J, Feldman SF, Lerner I, Rance B, Neuraz A, Burgun A. MedExt: combining expert knowledge and deep learning for medication extraction from French clinical texts. 2020. 23 Jan. 2020.

48. WHO Collaborating Centre for Drug Statistics Methodology. ATC/DDD Index 2021. 2020.

49. Von Elm E, Altman DG, Egger M, Pocock SJ, Gøtzsche PC, Vandenbroucke JP. The Strengthening the Reporting of Observational Studies in Epidemiology (STROBE) statement: guidelines for reporting observational studies. Ann Intern Med. 2007; 147:573-577.

50. Hayasaka Y, Purgato M, Magni LR, et al. Dose equivalents of antidepressants: Evidence-based recommendations from randomized controlled trials. Journal of affective disorders. Jul. 15, 2015,;180:179-184.

51. RECOVERY Collaborative Group, Horby P, Mafham M, Linsell L, Bell JL, Staplin N, Emberson JR, Wiselka M, Ustianowski A, Elmahi E, Prudon B, Whitehouse T, Felton T, Williams J, Faccenda J, Underwood J, Baillie JK, Chappell LC, Faust SN, Jaki T, Jeffery K, Lim WS, Montgomery A, Rowan K, Tarning J, Watson JA, White NJ, Juszczak E, Haynes R, Landray MJ. Effect of Hydroxychloroquine in Hospitalized Patients with Covid-19. N Engl J Med. 2020 Nov 19;383(21):2030-2040. doi: 10.1056/NEJMoa2022926. Epub 2020 Oct 8. PMID: 33031652; PMCID: PMC7556338.

52. Beigel JH, Tomashek KM, Dodd LE, Mehta AK, Zingman BS, Kalil AC, Hohmann E, Chu HY, Luetkemeyer A, Kline S, Lopez de Castilla D, Finberg RW, Dierberg K, Tapson V, Hsieh L, Patterson TF, Paredes R, Sweeney DA, Short WR, Touloumi G, Lye DC, Ohmagari N, Oh MD, Ruiz-Palacios GM, Benfield T, Fätkenheuer G, Kortepeter MG, Atmar RL, Creech CB, Lundgren J, Babiker AG, Pett S, Neaton JD, Burgess TH, Bonnett T, Green M, Makowski M, Osinusi A, Nayak S, Lane HC; ACTT-1 Study Group Members. Remdesivir for the Treatment of Covid-19 - Final Report. N Engl J Med. 2020 Nov 5;383(19):1813-1826. doi: 10.1056/NEJMoa2007764. Epub 2020 Oct 8. PMID: 32445440; PMCID: PMC7262788.

53. RECOVERY Collaborative Group. Azithromycin in patients admitted to hospital with COVID-19 (RECOVERY): a randomised, controlled, open-label, platform trial. Lancet. 2021 Feb 2:S0140-6736(21)00149-5. doi: 10.1016/S0140-6736(21)00149-5. Epub ahead of print. PMID: 33545096.

54. Choy KT, Wong AY, Kaewpreedee P, Sia SF, Chen D, Hui KPY, Chu DKW, Chan MCW, Cheung PP, Huang X, Peiris M, Yen HL. Remdesivir, lopinavir, emetine, and homoharringtonine inhibit SARS-CoV-2 replication in vitro. Antiviral Res. 2020 Jun;178:104786. doi: 10. 1016/j.antiviral.2020.104786. Epub 2020 Apr 3. PMID: 32251767; PMCID: PMC7127386.

55. Agostini ML, Andres EL, Sims AC, et al. Coronavirus Susceptibility to the Antiviral Remdesivir (GS-5734) Is Mediated by the Viral Polymerase and the Proofreading Exoribonuclease. mBio. 2018;9(2):e00221-18. Published 2018 Mar 6. doi:10.1128/mBio.00221-18

56. Körner RW, Majjouti M, Alcazar MAA, Mahabir E. Of Mice and Men: The Coronavirus MHV and Mouse Models as a Translational Approach to Understand SARS-CoV-2. Viruses. 2020; 12(8):880. Published 2020 Aug 12. doi:10.3390/v12080880

57. Marin-Corral J. et al, Metabolic Signatures Associated with Severity in Hospitalized COVID-19 Patients, Int J Mol Sci. 2021 May; 22(9): 4794.

58. Diaz Nebreda A, Zappia CD, Rodriguez González A, Sahores A, Sosa M, Burghi V, Monczor F, Davio C, Fernández N, Shayo C. Involvement of histamine H 1 and H 2 receptor inverse agonists in receptor’s crossregulation. Eur J Pharmacol. 2019 Mar 15;847:42-52

1. A Functional Inhibitor of Acid SphingoMyelinAse (FIASMA) agent for use for preventing or treating a viral infection due to at least one betacoronavirus in a patient in need thereof.

2. The FIASMA agent for use according to claim 1, wherein the betacoronavirus is a Severe Acute Respiratory Syndrome-related coronavirus (SARS-CoV), in particular SARS-CoV-2.

3. The FIASMA agent for use according to claim 2, for preventing or treating patients suffering from a symptomatic COVID-19 disease, and in particular from a strong or severe symptomatic COVID-19 disease.

4. The FIASMA agent for use according to any one of claims 1 to 3, for preventing and/or treating acute respiratory distress syndrome associated to said viral infection.

5. The FIASMA agent for use according to any one of claims 1 to 4, for lowering the risk of intubation or for increasing the survival rate of patients infected by said betacoronavirus.

6. The FIASMA agent for use according to any one of claims 1 to 5, wherein said agent can directly inhibit the activity of the ASM enzyme (EC 3.1.4.12).

7. The FIASMA agent for use according to any one of claims 1 to 5, wherein said agent can reduce the ceramide level at the surface of epithelial cells.

8. The FIASMA agent for use according to any one of claims 1 to 5, wherein said agent can indirectly inhibit the activity of the ASM enzyme (EC 3.1.4.12), for example by displacing the enzyme from lysosomal membranes and cause their degradation.

9. The FIASMA agent for use according to any one of claims 1 to 5, wherein said agent can directly neutralize surficial ceramide by interacting with same.

10. The FIASMA agent for use according to any one of claims 1 to 5, said FIASMA agent being an antidepressant.

11. The FIASMA agent for use according to claim 10, said antidepressant being fluoxetine or fluvoxamine or a pharmaceutical salt thereof.

12. The FIASMA agent for use according to claim 10, wherein said antidepressant is chosen in the group consisting of: Amitriptyline, Sertraline, Clomipramine, Duloxetine, Paroxetine, Escitalopram, Citalopram, Venlafaxine and Mirtazapine, or a pharmaceutical salt thereof.

13. The FIASMA agent for use according to claim 10, wherein said antidepressant is chosen in the group consisting of: Protriptyline, Nortriptyline, Maprotiline, Trimipramine, Desipramine, Lofepramine, Imipramine, and Doxepin, or a pharmaceutical salt thereof.

14. The FIASMA agent for use according to claim 10, wherein said antidepressant is chosen in the group consisting of: fluoxetine, citalopram, escitalopram, clomipramine, duloxetine, mirtazapine, venlafaxine, sertraline, paroxetine, and fluvoxamine, or a pharmaceutical salt thereof.

15. The FIASMA agent for use according to any of claim 1 to 14, wherein it is administered at an effective Fluoxetine-equivalent dose comprised between about 5 mg/day and about 60 mg/day, preferably between about 20 mg/day and about 40 mg/day, in patients in need thereof.

16. The FIASMA agent for use according to any one of claims 1 to 5, wherein said agent is an antipsychotic selected in the group consisting of: Triflupromazine, Trifluoperazine, Thioridazin, Sertindole, Fluphenazine, Flupenthixol, Perphenazine, Aripiprazole, Penfluridol Chlorprothixene, Pimozide, Promazine, and Chlorpromazine or a pharmaceutical salt thereof.

17. The FIASMA agent for use according to any one of claims 1 to 5, wherein said agent is an anti-histaminic agent selected in the group consisting of: Astemizole, Clemastine, Cyproheptadine, Desloratadine, Loratadine, Promethazine, Hydroxyzine and Terfenadine, or a pharmaceutical salt thereof.

18. The FIASMA agent for use according to any one of claims 1 to 5, wherein said agent is an anti-histaminic agent selected in the group consisting of: Mebhydrolin and Pimethixene, or a pharmaceutical salt thereof.

19. The FIASMA agent for use according to any of claim 1-5, wherein it is chosen in the group consisting of: Cinnarizine, Flunarizine, Benztropine, Biperidene, Profenamine, Emetine, Quinacrine, Amlodipine, Bepridil, Fendiline, Mibefradil, Perhexiline, Carvedilol, Amiodarone, Aprindine, Alverine, Camylofin, Dicycloverine, Mebeverine, Cinnarizine, Flunarizine, Conessine, Solasodine, Tomatidine, Dilazep, Suloctidil, Cloperastine, Loperamide, Clofazimine, Tamoxifen and Cyclobenzaprine, or a pharmaceutical salt thereof.

20. The FIASMA agent for use according to any of claim 1-5, wherein it is ambroxol.

21. The FIASMA agent for use according to any of claim 1-5, wherein it is chosen in the group consisting of: Diazepam, Fluoxetine, Fluvoxamine, Amitriptyline, Sertraline, Clomipramine, Duloxetine, Paroxetine, Escitalopram, Citalopram, Venlafaxine, Mirtazapine, Dosulepin, Vortioxetin, Milnacipran, Protriptyline, Nortriptyline, Maprotiline, Trimipramine, Desipramine, Lofepramine, Imipramine, Doxepin, Triflupromazine, Trifluoperazine, Thioridazin, Sertindole, Fluphenazine, Flupenthixol, Perphenazine, Aripiprazole, Penfluridol Chlorprothixene, Pimozide, Promazine, Chlorpromazine, Ambroxol, Astemizole, Clemastine, Cyproheptadine, Desloratadine, Loratadine, Promethazine, Hydroxyzine, Terfenadine, Mebhydrolin, Pimethixene, Cinnarizine, Flunarizine, Benztropine, Biperidene, Profenamine, Emetine, Quinacrine, Amlodipine, Bepridil, Fendiline, Mibefradil, Perhexiline, Carvedilol, Amiodarone, Aprindine, Alverine, Camylofin, Dicycloverine, Dicyclomine, Mebeverine, Cinnarizine, Flunarizine, Conessine, Solasodine, Tomatidine, Dilazep, Suloctidil, Clomifene, Cloperastine, Loperamide, Clofazimine, Tamoxifen, and Cyclobenzaprine, or a pharmaceutical salt thereof.
